(19) United States
(12) Patent Application Publication
(10) Pub. No.: US 2023/0386045 A1
(43) Pub. Date: Nov. 30, 2023

(54) SYSTEMS AND METHODS FOR AUTOMATED TEETH TRACKING

(71) Applicant: SDC U.S. SmilePay SPV, Nashville, TN (US)

(72) Inventors: Ryan Amelon, Nashville, TN (US); Saul Kohn, Nashville, TN (US); Aleksey Gurtovoy, Nashville, TN (US)

(73) Assignee: SDC U.S. SmilePay SPV, Nashville, TN (US)

(21) Appl. No.: 18/202,646

(22) Filed: May 26, 2023

Related U.S. Application Data
(60) Provisional application No. 63/346,646, filed on May 27, 2022.

(51) Int. Cl. G06T 7/11; G06T 7/136; A61C 7/00; G06T 7/55

(52) U.S. Cl. CPC G06T 7/11; G06T 7/136; A61C 7/002; G06T 7/55; G06T 2207/20081

(57) ABSTRACT

Systems and methods disclosed herein include a processor and a non-transitory computer readable medium containing instructions that when executed by the processor causes the processor to receive an image representing a first portion of a mouth of a user, segment the image to generate segmented regions of teeth present in the image, generate an imaging record by mapping the segmented regions of teeth present in the image to a template model where the imaging record indicates regions of the teeth that remain to be captured, and provide feedback identifying the regions of the teeth that remain to be captured based on the imaging record.

### CROSS-REFERENCE TO RELATED APPLICATIONS

This application claims the benefit of and priority to U.S. Provisional Patent Application No. 63/346,646, filed on May 27, 2022, which is incorporated herein by reference in its entirety and for all purposes.

### TECHNICAL FIELD

The present disclosure relates generally to the field of dentistry, and more specifically to systems and methods for automatically tracking teeth to determine which teeth and how much of each tooth are identified in images, and using the identified teeth for dental or orthodontic care.

### BACKGROUND

Processing images of a patient's teeth for purposes of dental or orthodontic care may require segmenting the teeth so that individual teeth can be identified and analyzed. However, typical segmentation processes are concerned with segmenting entire teeth. While segmenting entire teeth is useful for indicating whether a tooth is present in any given image, such processes do not determine how much of a tooth is actually visible in any given image.

### SUMMARY

In one aspect, this disclosure is directed to a system. The system includes a processor and a non-transitory computer readable medium containing instructions that when executed by the processor causes the processor to receive an image representing a first portion of a mouth of a user, segment the image to generate segmented regions of teeth present in the image, generate an imaging record by mapping the segmented regions of teeth present in the image to a template model where the imaging record indicates regions of the teeth that remain to be captured, and provide feedback identifying the regions of the teeth that remain to be captured based on the imaging record.

In another aspect, this disclosure is directed to a computer-implemented method. The method includes receiving, by one or more computer servers having a processor and non-transitory machine readable media, an image representing a first portion of a mouth of a user. The method further includes segmenting, by the one or more computer servers, the image to generate segmented regions of teeth present in the image. The method further includes generating, by the one or more computer servers, an imaging record by mapping the segmented regions of teeth present in the image to a template model where the imaging record indicates regions of the teeth that remain to be captured. The method further includes providing, by the one or more computer servers, feedback identifying the regions of the teeth that remain to be captured based on the imaging record.

In another aspect, this disclosure is directed to a computer-implemented method. The method includes receiving, by a teeth tracking application operating on a user device associated with a user and from a capture device of the user device, an image representing a first portion of a mouth of a user. The method further includes segmenting, by the teeth tracking application, the image to generate segmented regions of teeth present in the image. The method further includes generating, by the teeth tracking application, an imaging record by mapping the segmented regions of teeth present in the image to a template model where the imaging record indicates regions of the teeth that remain to be captured. The method further includes providing, by the teeth tracking application on a display of the user device, feedback identifying the regions of the teeth that remain to be captured based on the imaging record.

Various other embodiments and aspects of the disclosure will become apparent based on the drawings and detailed description of the following disclosure.

### BRIEF DESCRIPTION OF THE DRAWINGS

FIG. 1 shows a block diagram of a computer-implemented system including a teeth tracking application utilizing a machine learning architecture, according to an illustrative embodiment.

FIG. 2 shows a block diagram of an example system using supervised learning that may be used to segment an image, according to an illustrative embodiment.

FIG. 3 shows a block diagram of a simplified neural network model, according to an illustrative embodiment.

FIG. 4 shows a machine learning model trained to establish dense correspondences between 2D images (e.g., images of teeth) and 3D models (e.g., the template), according to an illustrative embodiment.

FIG. 5 shows a block diagram of an example system using semi-supervised learning that may be used to map pixels of a 2D image to a 3D surface, according to an illustrative embodiment.

FIG. 6 shows an example of a captured image being mapped to a template model, according to an illustrative embodiment.

FIG. 7 shows an example display of a template model and an indication of the views of the tooth that have been captured, according to an illustrative embodiment.

FIG. 8 shows a user receiving mirrored user feedback, according to an illustrative embodiment.

FIG. 9 shows an agent-based feedback selection model, according to an illustrative embodiment.

FIG. 10 shows examples of types of user feedback and a corresponding user script for each type of user feedback, according to an illustrative embodiment.

FIG. 11 shows an interactive communication flow utilizing the teeth tracking application to improve a quality of an image, according to an illustrative embodiment.

FIG. 12 shows an example of interactive communication resulting from the implementation of the machine learning architecture of FIG. 11, according to an illustrative embodiment.

FIG. 13 shows an example of interactive communication resulting from the implementation of the machine learning architecture of FIG. 11, according to an illustrative embodiment.

FIG. 14 shows another example of interactive communication resulting from the implementation of the machine learning architecture of FIG. 11, according to an illustrative embodiment.

FIG. 15 shows an interactive communication flow utilizing the teeth tracking application to track teeth or portions of teeth across images, according to an illustrative embodiment.

FIGS. 16A-16B show examples of a user approving/acknowledging a treatment plan determined by a downstream application, according to an illustrative embodiment.

### DETAILED DESCRIPTION

The present disclosure is directed to systems and methods for automatically cumulatively tracking teeth in images to determine which teeth, and how much of each tooth, are present in the images. Given images (e.g. a video or pictures) acquired of a user's teeth, a teeth tracking application automatically detects teeth regions in the images and tracks the cumulatively captured teeth regions. Based on the cumulative data of the captured teeth, the captured teeth are displayed to a user. In some embodiments, teeth remaining to be captured are also or alternatively displayed to the user. While “teeth regions” is used through the application for ease of reference, it will be appreciated that “teeth regions” could alternatively be “mouth regions” of the user's mouth and the “mouth regions” could include any part of the user's mouth, such as teeth, lips, tongue, palates, gums, uvula, or other features of the mouth. For example, the teeth tracking application can be configured to monitor the progression of a patient's dental health over time, such as an individual tooth, or groups of teeth like molars, incisors, or even specific sections of a tooth. In another example, the teeth tracking application can be configured to track the health of gums, such as receding gum lines or changes in gum color. In yet another example, the teeth tracking application can be configured to track and monitor conditions related to oral mucosa, such as monitoring the movement and shape of the tongue and uvula during speech and swallowing, aiding in identifying conditions like dysarthria or dysphagia.

The teeth tracking application is used to record captured images of teeth, but also to capture and track the various views (angles) of captured regions of teeth. The teeth tracking application may be used to determine whether the pictures or a captured video stream has acquired enough coverage of teeth to be useful to various downstream applications. In an example, the teeth tracking application may track captured images of teeth (e.g., the bottom of a tooth, the top of a tooth, the side of a tooth) such that enough of the tooth is captured for downstream applications, where each downstream application may require various teeth to be captured, various views of teeth to be captured, and/or various image qualities.

The systems and processes described herein have many benefits over existing systems and processes. For example, the teeth tracking application improves a user experience associated with capturing high quality images of the user's dentition by reducing costs and time that would otherwise be incurred as part of the user needing to otherwise visit trained professionals by communicating relevant feedback to the user in a user-friendly way. The interactive user-specific feedback provided to the user improves the quality of images captured while decreasing the time and effort that the user spends before capturing high quality images. For instance, the characteristics of the image (e.g., contrast, sharpness, brightness, blur) and the content of the image (visibility of teeth, mouth angle, tongue position) are evaluated by the teeth tracking application to determine whether the captured image is a high quality image. The interactive user-specific feedback also identifies high quality images captured by the user or images remaining to be captured by the user depending on the requirements of downstream processes. Communicating to the user the particular teeth that have been captured and/or the remaining teeth to be captured reduces the need for future communication to/from the user. For example, the user experience is improved by minimizing the need for additional images to be captured in response to a downstream process not having a particular view of a particular tooth. The embodiments also improve the user experience by communicating user-specific feedback. That is, the feedback is provided by available user hardware, is directed to facilitating a particular user in capturing a high quality image of a particular tooth/view in response to received images, and it is heterogeneously communicated to the user according to user preferences. Communicating user-specific feedback reduces computational resources consumed by a system that would otherwise communicate general feedback by limiting the number of iterations necessary to capture a high quality image of the user's dentition. For example, computational resources are conserved by limiting general communications and/or standard feedback to the user in an attempt to guide the user to capture a high quality image. Moreover, computational resources are conserved by limiting instructions to the user when the user has already performed necessary actions (e.g., capturing an image of a specific tooth or view/angle of the specific tooth that the user has already captured).

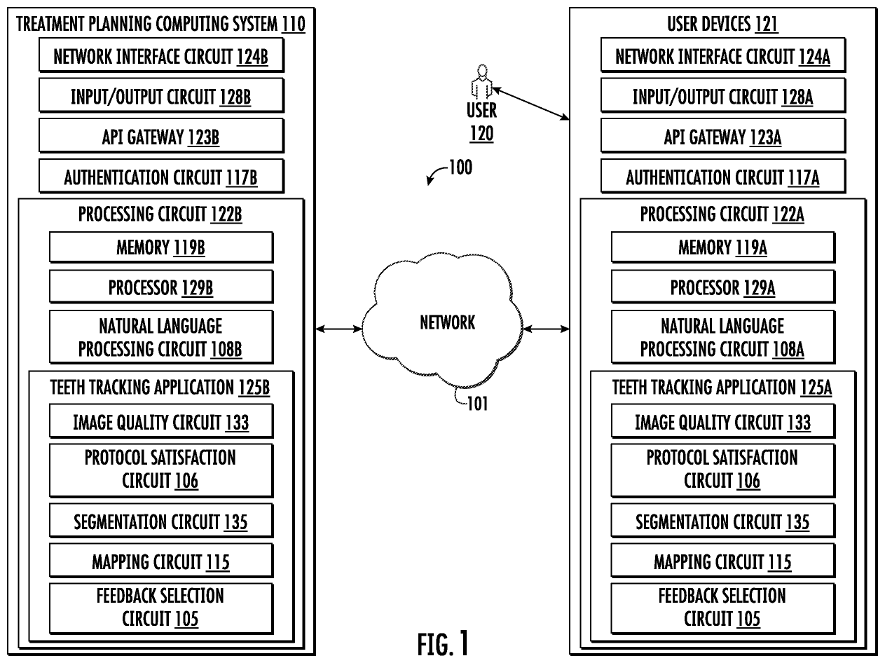

Referring now to FIG. 1, a block diagram of a computer-implemented system 100 including a teeth tracking application utilizing a machine learning architecture is shown, according to an embodiment. The system 100 includes user device 121 and treatment planning computing system 110. Devices and components in FIG. 1 can be added, deleted, integrated, separated, and/or rearranged in various embodiments of the disclosed inventions. In some embodiments, operations may be performed on the user device 121. For example, the user device 121 may analyze the captured data. However, in other embodiments, operations may be performed on the treatment planning computing system 110. For example, the user device 121 may capture an image, subsequently transmit the image to the treatment planning computing system 110 for processing. For ease of description, the treatment planning computing system 110 is described as executing the teeth tracking application. But it should be appreciated that the user device 121 and/or some combination of the user device 121 and the treatment planning computing system 110 may execute one or more operations of the teeth tracking application. Components of the user device 121 and/or treatment planning computing system 110 may be locally installed (on the user device 121 and/or treatment planning computing system 110), and/or may be remotely accessible (e.g., via a browser based interface or a cloud system).

The various systems and devices may be communicatively and operatively coupled through a network 101. Network 101 may permit the direct or indirect exchange of data, values, instructions, messages, and the like (represented by the arrows in FIG. 1). The network 101 may include one or more of the Internet, cellular network, Wi-Fi, Wi-max, a proprietary network, or any other type of wired or wireless network of a combination of wired or wireless networks. The network 101 may facilitate communication between the respective components of the system 100, as described in greater detail below.

The user 120 may be any person using the user device 121. Such a user 120 may be a potential customer, a customer, client, patient, or account holder of an account stored in treatment planning computing system 110 or may be a guest user with no existing account. The user device 121 includes any type of electronic device that a user 120 can access to communicate with the treatment planning computing system 110. For example, the user device 121 may include watches (e.g., a smart watch), and computing devices (e.g., laptops, desktops, personal digital assistants (PDAs), mobile devices (e.g., smart phones)). In some embodiments, a different device may be configured to capture images (e.g. a video or pictures) of the user 120. The different device may communicate the captured images of the user 120 to the user device 121 for subsequent processing. For instance, the user device 121 may execute teeth tracking application 125A using the captured images of the user 120. Additionally or alternatively, the user device 121 may transmit the captured images of the user 120 to the treatment planning computing system 110 such that the treatment planning computing system 110 may execute teeth tracking application 125B (instead of, or in addition to the teeth tracking application 125A executed on the user device 121).

The treatment planning computing system 110 may be associated with or operated by a dental institution (e.g., a dentist or an orthodontist, a clinic, a dental hardware manufacturer). The treatment planning computing system 110 may maintain accounts held by the user 120, such as personal information accounts (patient history, patient issues, patient preferences, patient characteristics). The treatment planning computing system 110 may include server computing systems, for example, comprising one or more networked computer servers having a processor and non-transitory machine readable media. In some embodiments, the treatment planning computing system 102 may include a plurality of servers, which may be located at a common location (e.g., a server bank) or may be distributed across a plurality of locations.

As shown, both the user device 121 and the treatment planning computing system 110 may include a network interface (e.g., network interface 124A at the user device 121 and network interface 124B at the treatment planning computing system 110, hereinafter referred to as “network interface 124”), a processing circuit (e.g., processing circuit 122A at the user device 121 and processing circuit 122B at the treatment planning computing system 110, hereinafter referred to as “processing circuit 122”), an input/output circuit (e.g., input/output circuit 128A at the user device 121 and input/output circuit 128B at the treatment planning computing system 110, hereinafter referred to as “input/output circuit 128”), an application programming interface (API) gateway (e.g., API gateway 123A at the user device 121 and API gateway 123B at the treatment planning computing system 110, hereinafter referred to as “API gateway 123”), and an authentication circuit (e.g., authentication circuit 117A at the user device 121 and authentication circuit 117B at the treatment planning computing system 110, hereinafter referred to as “authentication circuit 117”). The processing circuit 122 may include a memory (e.g., memory 119A at the user device 121 and memory 119B at the treatment planning computing system 110, hereinafter referred to as “memory 119”), a processor (e.g., processor 129A at the user device 121 and processor 129B at the treatment planning computing system 110, hereinafter referred to as “processor 129”), a natural language processing circuit (e.g., natural language processing circuit 108A at the user device 121 and natural language processing circuit 119B at the treatment planning computing system 110, hereinafter referred to as “NLP circuit 108”), and a teeth tracking application (e.g., teeth tracking application 125A at the user device 121 and teeth tracking application 125B at the treatment planning computing system 110, hereinafter referred to as “teeth tracking application 125”). In some embodiments, the teeth tracking application may be an application as part of (e.g., incorporated into) a different application of the user device 121 or the treatment planning computing system 110.

The network interface circuit 124 may be adapted for and configured to establish a communication session via the network 101 between the user device 121 and the treatment planning computing system 110. The network interface circuit 124 includes programming and/or hardware-based components that connect the user device 121 and/or treatment planning computing system 110 to the network 101. For example, the network interface circuit 124 may include any combination of a wireless network transceiver (e.g., a cellular modem, a Bluetooth transceiver, a Wi-Fi transceiver) and/or a wired network transceiver (e.g., an Ethernet transceiver). In some arrangements, the network interface circuit 124 includes the hardware and machine-readable media structured to support communication over multiple channels of data communication (e.g., wireless, Bluetooth, near-field communication, etc.).

Further, in some arrangements, the network interface circuit 124 includes cryptography module(s) to establish a secure communication session (e.g., using the IPSec protocol or similar) in which data communicated over the session is encrypted and securely transmitted. In this regard, personal data (or other types of data) may be encrypted and transmitted to prevent or substantially prevent the threat of hacking or unwanted sharing of information.

To support the features of the user device 121 and/or treatment planning computing system 110, the network interface circuit 124 provides a relatively high-speed link to the network 101, which may be any combination of a local area network (LAN), the Internet, or any other suitable communications network, directly or through another interface.

The input/output circuit 128A at the user device 121 may be configured to receive communication from a user 120 and provide outputs to the user 120. Similarly, the input/output circuit 128B at the treatment planning computing system 110 may be configured to receive communication from an administrator (or other user such as a medical professional, such as a dentist, orthodontist, dental technician, or administrator) and provide output to the user. For example, the input/output circuit 128 may capture user responses based on a selection from a predetermined list of user inputs (e.g., drop down menu, slider, buttons), an interaction with a microphone on the user device 121, or an interaction with a graphical user interface (GUI) displayed on the user device 121, an interaction with a light sensor, an interaction with an accelerometer, and/or an interaction with a camera. For example, a user 120 using the user device 121 may capture an image of the user 120 using a camera. In some embodiments, the camera may be a “front-facing camera.” For example, a front-facing camera may be a camera that is on a front of the user device 121 such that the user 120 may view the display of the user device 121 while facing the camera. In other embodiments, the camera may be a “rear-facing camera.” A rear-facing camera may be a camera that is on a back of the user device 121 such that the user 120 may not view the display of the user device 121 and face the rear-facing camera at the same time. In some embodiments, the rear-facing camera is the same type of camera as the front-facing camera. In other embodiments, the rear-facing camera may be configured to take higher quality images and/or record higher quality video streams than the front-facing camera. The image of the user captured by the camera (either the front-facing camera or the rear-facing camera) may be ingested by the user device 121 using the input/output circuit 128. Similarly, a user device 121 may interact with the light sensors on the user device such that the light sensors can collect data to determine whether the user device 121 is facing light. Further, a user 120 may interact with the accelerometer such that the accelerometer may interpret measurement data to determine whether the user 120 is shaking the user device 121, and/or may provide feedback regarding the orientation of the device and whether the user 120 is modifying the orientation of the user device 121. Feedback associated with the captured image may be output to the user using the input/output circuit 128. For example, the teeth tracking application 125 may provide instructions to generate audible feedback to the user using speakers on the user device 121. Additionally or alternatively, the user 120 may interact with the GUI executed by the user device 121 using the user's 120 voice, a keyboard/mouse (or other hardware), and/or a touch screen.

In some embodiments, the digital information captured and/or ingested by the input/output circuit 128 can be used as input for machine learning models deployed in the teeth tracking application 125. The machine learning models can be trained on dental imagery, enabling the models to identify and track various features within the oral cavity. In some embodiments, other sensors such as a light sensor and accelerometer can be used by the teeth tracking application 125 to improve the digital information captured. Thus, it should be understood that machine learning models, used in conjunction with other sensory input such as light sensors and accelerometers, can improve the image quality for the dental tracking application 125. For example, ambient light conditions captured by light sensors, when combined with the image analysis of the machine learning models, can be used to adjust various photographic parameters to optimize the image quality. In particular, in low-light conditions, the teeth tracking application 125 could increase the exposure or ISO settings on the camera, allowing more light onto the camera sensor and producing a brighter, clearer image. On the other hand, in brightly lit conditions, the teeth tracking application 125 can lower the exposure or ISO settings to prevent overexposure and maintain the clarity of the details. In another example, device orientation and movement data collected by one or more accelerators, can be used by the machine learning models to enhance image stability and focus. In particular, if the user moves or shakes the device or changes its orientation while capturing an image, the application can use this data to digitally stabilize the image and maintain the focus on the area of interest within the mouth region. For instance, if the user is moving the device towards a particular tooth, the application could adjust the camera's focal point to keep that tooth in sharp focus. Thus, the accelerometer-assisted focus adjustment can be used by the machine learning model to track specific teeth or mouth regions.

The API gateway 123 may be configured to facilitate the transmission, receipt, authentication, data retrieval, and/or exchange of data between the user device 121, and/or treatment planning computing system 110.

Generally, an API is a software-to-software interface that allows a first computing system of a first entity (e.g., the user device 121) to utilize a defined set of resources of a second (external) computing system of a second entity (e.g., the treatment planning computing system 110, or a third party) to, for example, access certain data and/or perform various functions. In such an arrangement, the information and functionality available to the first computing system is defined, limited, or otherwise restricted by the second computing system. To utilize an API of the second computing system, the first computing system may execute one or more APIs or API protocols to make an API “call” to (e.g., generate an API request that is transmitted to) the second computing system. The API call may be accompanied by a security or access token or other data to authenticate the first computing system and/or a particular user 120. The API call may also be accompanied by certain data/inputs to facilitate the utilization or implementation of the resources of the second computing system, such as data identifying users 120 (e.g., name, identification number, biometric data), accounts, dates, functionalities, tasks, etc.

The API gateway 123 in the user device 121 provides various functionality to other systems and devices (e.g., treatment planning computing system 110) through APIs by accepting API calls via the API gateway 123. The API calls may be generated via an API engine of a system or device to, for example, make a request from another system or device.

For example, the teeth tracking application 125B at the treatment planning computing system 110 and/or a downstream application operating on the treatment planning computing system 110 may use the API gateway 123B to communicate with the teeth tracking application 125A. The communication may include commands to control the teeth tracking application 125A. For example, a circuit of the teeth tracking application 125B (e.g., the segmentation circuit 135, the feedback selection circuit 105) may result in (or produce) an output that may start/stop a process (e.g., start or stop an image capture process). Similarly, upon the downstream application or teeth tracking application 125B determining a certain result (e.g., a threshold number of tracked teeth), the downstream application and/or teeth tracking application 125B may send a command to the teeth tracking application 125A via the API gateway to perform a certain operation (e.g., turn off an active camera at the user device 121).

The authentication circuit 117 of the treatment planning computing system 110 may be or include any device(s), component(s), circuit(s), or other combination of hardware components designed or implemented configured to authenticate the user 120 by authenticating information received by the user device 121. The authentication circuit 117 authenticates a user 120 as having a user account associated with the treatment planning computing system 110 (and/or the teeth tracking application 125). The user account (or user profile) may be an account associated with a particular user and identify user information and/or medical information. For example, the user account may identify the user's name, email address, home address, biometric data, user name, passwords, feedback preferences, gender, age, purchase history (e.g., purchased aligners), treatment history (e.g., saved treatment plans and any associated information such as initial teeth positions and/or final teeth positions), dental conditions, user specific template models (as discussed herein) and/or characteristics. Characteristics may include characteristics of the user's detention. For example, a characteristic of the user's detention may be that the user is missing one or more teeth (or particular regions of teeth). Another characteristic of the user's detention may be that the user's teeth are bejeweled.

In some embodiments, the authentication circuit 117 may prompt the user 120 to enter user 120 credentials (e.g., username, password, security questions, and biometric information such as fingerprints or facial recognition). The authentication circuit 117 may look up and match the information entered by the user 120 to stored/retrieved user 120 information in memory 119. For example, memory 119 may contain a lookup table matching user 120 authentication information (e.g., name, home address, IP address, MAC address, phone number, biometric data, passwords, usernames) to a particular user account.

The processing circuit 122 may include at least memory 119 and a processor 129. The memory 119 includes one or more memory devices (e.g., RAM, NVRAM, ROM, Flash Memory, hard disk storage) that store data and/or computer code for facilitating the various processes described herein. The memory 119 may be or include tangible, non-transient volatile memory and/or non-volatile memory. The memory 119 stores at least portions of instructions and data for execution by the processor 129 to control the processing circuit 122. For example, memory 119 may serve as a repository for user 120 accounts (e.g., storing user 120 name, email address, physical address, phone number, medical history), training data, thresholds, weights, and the like for the machine learning models. In other arrangements, these and other functions of the memory 119 are stored in a remote database.

The processor 129 may be implemented as a general-purpose processor, an application specific integrated circuit (ASIC), one or more field programmable gate arrays (FPGAs), a digital signal processor (DSP), a group of processing components, or other suitable electronic processing components.

The NLP circuit 108 in the processing circuit 112 may be or include any device(s), component(s), circuit(s), or other combination of hardware components designed or implemented to determine information extracted from an audio signal from the user 120. For example, the NLP circuit 108 may be used to interpret user inputs when the user 120 is interacting with the teeth tracking application 125 orally. For instance, the user 120 may hold the user device 121 (e.g., at a particular position in air) and speak into a microphone or other component of the input/output circuit 128 on the user device 121. In an example, the user 120 may request that the teeth tracking application 125 repeat the user feedback. In some configurations, the NLP circuit 108 may parse the audio signal into audio frames containing portions of audio data. The frames may be portions or segments of the audio signal having a fixed length across the time series, where the length of the frames may be pre-established or dynamically determined.

The NLP circuit 108 may also transform the audio data into a different representation. For example, the NLP circuit 108 initially generates and represents the audio signal and frames (and optionally sub-frames) according to a time domain. The NLP circuit 108 transforms the frames (initially in the time domain) to a frequency domain or spectrogram representation, representing the energy associated with the frequency components of the audio signal in each of the frames, thereby generating a transformed representation. In some implementations, the NLP circuit 108 executes a Fast-Fourier Transform (FFT) operation of the frames to transform the audio data in the time domain to the frequency domain. For each frame (or sub-frame), the NLP circuit 108 may perform a simple scaling operation so that the frame occupies the range [−1, 1] of measurable energy.

In some implementations, the NLP circuit 108 may employ a scaling function to accentuate aspects of the speech spectrum (e.g., spectrogram representation). The speech spectrum, and in particular the voiced speech, will decay at higher frequencies. The scaling function beneficially accentuates the voiced speech such that the voice speech is differentiated from background noise in the audio signal. The NLP circuit 108 may perform an exponentiation operation on the array resulting from the FFT transformation to further distinguish the speech in the audio signal from background noise. The NLP circuit 108 may employ automatic speech recognition and/or natural language processing algorithms to interpret the audio signal.

The user device 121 and/or treatment planning computing system 110 are configured to run a variety of application programs and store associated data in a database of the memory 119. One such application executed by the user device 121 and/or treatment planning computing system 110 using the processing circuit 122 may be the teeth tracking application 125. The teeth tracking application 125 is structured to guide a user (e.g., user 120 using a user device 121) to capture images of teeth (including portions of teeth) and one or more corresponding views of the teeth for one or more downstream applications. The teeth tracking application guides the user 120 in capturing images of teeth at various views required for one or more downstream applications by tracking the previously captured images of teeth at various views.

The teeth tracking application 125 automatically detects a view of a teeth region and/or detects teeth regions in captured images (e.g., videos or pictures) of a user's dentition and tracks the cumulatively captured teeth regions corresponding views of the teeth regions. The teeth tracking application 125 may implicitly guide the user to capture images of teeth in various views. For example, the teeth tracking application 125 may display, to the user via the user device 121, the received images of teeth and/or the remaining teeth (or corresponding views of teeth that need to be captured). In this way, the user is implicitly guided because the user can determine how to position themselves/the camera to capture one or more remaining teeth at one or more remaining views using the display indicating the teeth captured by the teeth tracking application.

The teeth tracking application 125 may also explicitly guide the user to capture images of teeth in various views. For example, the teeth tracking application 125 may display, to the user via the user device, instructions (e.g., feedback) on how the user can position the user device 121, how the user can position themselves (e.g., their jaw, their tongue, their lips), and/or some combination, to subsequently capture images of any remaining teeth (or corresponding views of the teeth). The feedback selected by the teeth tracking application 125 to guide the user to capture teeth in particular views minimizes the number of attempts (or duration of time) that the user 120 spends attempting to capture high quality images of remaining teeth in various views, minimizes the effort required by the user 120 to capture high quality images of remaining teeth in various views, and/or improves the user 120 experience with the teeth tracking application 125.

In another example, teeth tracking application 125 can present a semi-transparent overlay that indicates the optimal position for the user's oral cavity in relation to the device's camera. This overlay, superimposed on the live camera feed, can visually guide the user 120 to align their mouth appropriately. In particular, it can track the average position of the teeth, adapting to the user's movements, provide real-time feedback, and assist the user 120 to correctly position themselves in relation to the overlay.

As described herein, the teeth tracking application 125 determines feedback that may be used to improve subsequently captured images. Accordingly, instead of filtering out low quality data (e.g., data that does not satisfy the image quality thresholds associated with the characteristics of the image and/or image quality thresholds associated with the content of the image), the teeth tracking application 125 uses the low quality data to determine feedback that may improve the quality of captured images and/or guide the user to capture images of any remaining teeth/views. The teeth tracking application 125 therefore improves the user experience by reducing the number of low quality images captured by the user (e.g., reducing the number of errors), and shortening the capturing acquisition times necessary to capture any remaining high quality images of teeth in various views.

The teeth tracking application 125 may also both implicitly and explicitly guide a user to capture high quality images of teeth at various views. For example, the teeth tracking application 125 may display, on user device 121, both the remaining teeth that need to be captured (and/or any remaining views of teeth that need to be captured) and instructions on how to capture high quality images of the remaining teeth/views.

In some embodiments, the teeth tracking application 125 may only display and track the high quality images of teeth. That is, if the teeth tracking application 125 determines that the received image of a tooth is not a high quality image, then the teeth tracking application 125 may not track/record the received image. Accordingly, the user will be implicitly/explicitly guided to re-capture the same tooth such that the user may capture a high quality image of the tooth.

The teeth tracking application 125 may utilize and/or instruct other circuits on the user device 121 such as components of the input/output circuit 128 (e.g., a display of the user device 121, a microphone on the user device 121, a front-facing camera on the user device 121, a rear-facing camera on the user device 121, etc.). For example, executing the teeth tracking application 125 may result in generating instructions to display on a user interface. A user interface displayed on the user device 121 may indicate a record of the captured teeth and corresponding views of those teeth, and/or a record of the remaining teeth to be captured, and corresponding views of those teeth. In some embodiments, data captured at the teeth tracking application 125A at the user device 121 is communicated to the teeth tracking application 125B at the treatment planning computing system 110.

The teeth tracking application 125 is a downloaded and installed application that includes program logic stored in a system memory (or other storage location) of the user device 121 that includes an image quality circuit 133 and protocol satisfaction circuit 106 configured to determine a quality of a captured image (both in terms of the characteristics of the image (e.g., blur, motion artifacts, lighting) and in terms of the content of the image (e.g., visibility of the teeth in the image)). The teeth tracking application 125 also includes a segmentation circuit 135 and a mapping circuit 115 configured to generate/update a running record of the images of teeth (and corresponding views of the teeth) that have been captured. The teeth tracking application 125 also includes a feedback selection circuit 105 configured to determine feedback that will improve the quality of a subsequently captured image (in terms of the characteristics of the subsequently captured image and/or in terms of the content of the subsequently captured image). The feedback selection circuit 105 is also configured to determine feedback that will guide the user to capture any remaining teeth (or portions of teeth) to be captured for a downstream application and/or any remaining views to be captured for the downstream application. In this embodiment, the image quality circuit 133, protocol satisfaction circuit 106, segmentation circuit 135, mapping circuit 115, and feedback selection circuit 105 are embodied as program logic (e.g., computer code, modules, etc.). The teeth tracking application 125A is communicably coupled via the network interface circuit 124A over the network 101 to the treatment planning computing system 110 and, particularly to the teeth tracking application 125B that may support at least certain processes and functionalities of the teeth tracking application 125A. Similarly, the teeth tracking application 125B is communicably coupled via the network interface circuit 124B over the network 101 to the user device 121, and particularly to the teeth tracking application 125A. In some embodiments, during download and installation, the teeth tracking application 125A is stored by the memory 119A of the user device 121 and selectively executable by the processor 129A. Similarly, in some embodiments, the teeth tracking application 125B is stored by the memory 119B of the treatment planning computing system 110 and selectively executable by the processor 129B. The program logic may configure the processor 129 (e.g., processor 129A of the user device 121 and processor 129B of the treatment planning computing system 110) to perform at least some of the functions discussed herein. In some embodiments the teeth tracking application 125 is a stand-alone application that may be downloaded and installed on the user device 121 and/or treatment planning computing system 110. In some embodiments, the teeth tracking application 125 may be a part of another application.

The depicted downloaded and installed configuration of the teeth tracking application 125 is not meant to be limiting. According to various embodiments, parts (e.g., modules, etc.) of the teeth tracking application 125 may be locally installed on the user device 121/treatment planning computing system 110 and/or may be remotely accessible (e.g., via a browser-based interface) from the user device 121/treatment planning computing system 110 (or other cloud system in association with the treatment planning computing system 110). In this regard and in another embodiment, the teeth tracking application 125 is a web-based application that may be accessed using a browser (e.g., an Internet browser provided on the user device). In still another embodiment, the teeth tracking application 125 is hard-coded into memory such as memory 119 of the user device 121/treatment planning computing system 110 (i.e., not downloaded for installation). In an alternate embodiment, the teeth tracking application 125 may be embodied as a “circuit” of the user device 121 as circuit is defined herein.

The operations performed by the teeth tracking application 125 may be executed at the user device 121, at the treatment planning computing system 110, and/or using some combination of the user device 121 and the treatment planning computing system 110. In a non-limiting example, the teeth tracking application 125 may be executed on the user device 121 such that the user 120 receives feedback related to guiding the user to capture remaining high quality regions of teeth and/or particular views of teeth in real time or near real time. The time associated with the user waiting to receive feedback is minimized (or reduced) because the feedback is being determined by the teeth tracking application 125A at the user device 121 (instead of being determined by the teeth tracking application 125B at the treatment planning computing system and needing to be transmitted to the user device 121). In another example, the teeth tracking application 125 may be executed both at the user device 121 (e.g., teeth tracking application 125A) and the treatment planning computing system 110 (e.g., teeth tracking application 125B). For example, a first teeth tracking application may be executed (e.g., the teeth tracking application 125A on the user device 121) to provide simple feedback, and a second teeth tracking application may be executed (e.g., the teeth tracking application 125B on the treatment planning computing system 110) to provide more sophisticated (and/or additional/supplemental) feedback to the user 120. In other implementations, the teeth tracking application 125 may be executed partially at the user device 121 and partially at the treatment planning computing system 110. Additionally or alternatively, the teeth tracking application 125 may be executed completely in the user device 121 (or treatment planning computing system 110), and in some implementations may be run subsequently at the treatment planning computing system 110 (or user device 121). In some implementations, the teeth tracking application 125A may run in parallel with the teeth tracking application 125B.

The teeth tracking application 125 includes an image quality circuit 133. The image quality circuit 133 may be or include any device(s), component(s), circuit(s), or other combination of hardware components designed or implemented to determine, identify, or otherwise evaluate the quality of a captured image (or a frame of a video data stream) with respect to the characteristics of the image. The quality of the image with respect to the characteristics of the image includes the visibility of the image (e.g., lightness/darkness in the image, shadows in the image), the contrast of the image, the saturation of an image, the sharpness of time image, and/or the blur of the image (e.g., motion artifacts), and/or the noise or distortion of an image, for instance.

The image quality circuit 133 may evaluate the quality of the image with respect to the characteristics of the image using a machine learning model. In one example implementation, the image quality circuit 133 may implement a Blind/Referenceless Image Spatial Quality Evaluator (BRISQUE) model. BRISQUE models are beneficial because the quality of an image affected by an unknown distortion can be evaluated. That is, the characteristics of the image (e.g., blue, contrast, brightness), do not need to be labeled/classified in a dataset before the quality of the image is determined. Further, BRISQUE can be performed quickly (e.g., in real time or near real time) because of its low computational complexity.

The BRISQUE model may be trained to evaluate the quality of an image using a dataset including clean images and distorted images (e.g., images affected by pixel noise). The BRISQUE model generates an image score using support vector regression. The training images may be normalized. In some implementations, mean subtracted contrast normalization may be employed to normalize the image. Features from the normalized image may be extracted and transformed into a higher dimension (e.g., mapping the data to a new dimension, employing the “kernel trick” using sigmoid kernels, polynomial kernels, radial basis function kernels, and the like) such that the data is linearly separable. Support vector regression trains/optimizes a hyperplane to model the feature inputs images. The hyperplane may be optimized by taking the gradient of a cost function (such as the hinge loss function) to maximize the margin of the hyperplane. Decision boundaries are determined (based on a tolerance) around the hyperplane.

In some implementations, the image quality circuit 133 can determine the characteristics of specific areas of the image. For example, the image quality circuit 133 may evaluate the image quality for different teeth in the image and/or different regions of teeth in the image. In some implementations, the image quality circuit 133 may determine, using the image quality score of specific areas of the image, whether the specific areas of the image are overexposed (or too dark). In one embodiment, the image quality circuit 133 can be applied to the whole image or parts of the image. For example, the image quality circuit 133 may receive an input from the segmentation circuit 135. The segmentation circuit 135, as discussed herein, is configured to segment areas of the mouth, regions of teeth, and the like. The image quality circuit 133 can be applied to each specific region to generate a quality score map of the image.

The teeth tracking application 125 also includes a protocol satisfaction circuit 106. The protocol satisfaction circuit 106 may be or include any device(s), component(s), circuit(s), or other combination of hardware components designed or implemented to evaluate the quality of a captured image (or a frame of a video data stream) with respect to the content of the image. The content of the image may include the prevalence, visibility, distinctiveness and/or relevance of various teeth and/or facial features in the image. That is, the protocol satisfaction circuit 106 evaluates what is or is not visible (e.g., an absence or presence), the extent (e.g., a degree) of the visibility, an angle, an orientation, and the like.

The protocol satisfaction circuit 106 may evaluate the prevalence, visibility, distinctiveness and/or relevance of facial features in the content of the image using object detection. For example, the protocol satisfaction circuit 106 may determine the angle, visibility, and/or orientation of a user's 120 facial features (e.g., teeth, lips, tongue, eyes, nose, mouth, chin) from the image.

The protocol satisfaction circuit 106 may employ any suitable object detection algorithm/model to detect the content of the image. In some embodiments, the protocol satisfaction circuit 106 may be applied to one or more parts of the image (e.g., one or more segmented images produced by segmentation circuit 135). One example object detection model of the protocol satisfaction circuit 106 that can operate in real time (or near real time) on images is the “you only look once” (YOLO) model. The YOLO model employs boundary boxes and class labels to identify objects in an image. The YOLO model is trained using a training dataset including classes identified in training images. For example, an image may be labeled with particular classes (e.g., facial features, such as chin, eyes, lips, nose, teeth) of objects detected in the image. In operation, the YOLO model partitions an image into a grid and determines whether each grid contains a portion of a boundary box and a corresponding likelihood of the boundary box belonging to the particular class.

In one implementation, the protocol satisfaction circuit 106 may employ photogrammetry to determine a position, orientation, size, and/or angle of a facial feature in the image. For instance, the roll, pitch, yaw and distance of the user's 120 head may be determined using photogrammetry or one or more other algorithms. Performing photogrammetry involves extracting three-dimensional (3D) measurements from the captured two-dimensional (2D) images.

In general, roll refers to the tilting motion of the head from side to side, pitch is the up and down nodding motion, while yaw is the side-to-side shaking motion. These orientations, coupled with the distance of the head from the camera, provide data for the protocol satisfaction circuit 106 to position and track teeth or mouth regions. However, in some embodiments, when it comes to the specifics of dental structures, considering the head's position may only provide an accurate representation of the upper jaw due to anatomical considerations. Thus, the lower jaw or mandible, which is mobile and independent of the upper jaw or maxilla, may need additional analysis and computation for accurate representations. To address this, the protocol satisfaction circuit 106 can employ a two-tier algorithmic approach to consider the unique dynamics of the lower jaw.

In some embodiments, the protocol satisfaction circuit 106 can assume that the roll and yaw are identical for both the upper and lower jaws, which is generally accurate given that any lateral or rotational movement would involve both jaws moving in conjunction. However, the pitch, representing the opening and closing of the mouth, may vary greatly between the jaws as it largely depends on the lower jaw's movement. In various embodiments, to model this, the lower jaw's movement is considered akin to a hinge joint, mirroring the biological articulation of the temporomandibular joint (TMJ). Based on this model, the protocol satisfaction circuit 106 computes the angular offset, which is the angle between a standard, fully closed position and the current position as observed in the mouth's bounding box. Accordingly, this calculated angle provides the pitch of the lower jaw, giving an understanding of its position relative to the upper jaw. In some embodiments, the protocol satisfaction circuit 106 can utilize inertial sensors for determining the head's roll, pitch, and yaw, and photogrammetry for capturing detailed structural information of the jaws. This dual-approach can also provide for accurate tracking and modeling of the upper and lower jaws' positions and movements.

The protocol satisfaction circuit 106 may perform photogrammetry by comparing known measurements of facial features with measurements of facial features in the image. The facial features measured in the image (e.g., including the lengths/sizes of the various facial features in the image) may include tooth measurements, lip size measurements, eye size measurements, chin size measurements, and the like. The known facial features may be average facial features (including teeth, chin, lips, eyes, nose) from one or more databases (e.g., treatment planning computing system 110 memory 119B) and/or from local memory 119A. The known facial features may also be particular measurements of a user (e.g., measured when the user 120 was at a medical professional's office) from local memory 119A and/or a database (e.g., treatment planning computing system 110 memory 119B). The protocol satisfaction circuit 106 compares the known measurements of facial features with dimensions/measurements of the facial features in the image to determine the position, orientation, size, and/or angle of the facial feature in the image.

The teeth tracking application 125 includes a segmentation circuit 135. The segmentation circuit 135 may be or include any device(s), component(s), circuit(s), or other combination of hardware components designed or implemented to determine, identify, or otherwise segment an image. As discussed herein, the image may be captured from a camera directly. In some embodiments, the image may be a single frame of a continuous stream of video data. An image is segmented when the image is marked to delineate a structure (e.g., a tooth, a region of a tooth, an arch). For example, a line may be drawn around a section of an image and labeled as being or being part of a particular tooth. Everything inside the line may be considered the tooth, while everything outside the line would not be considered the tooth.

The segmentation circuit 135 may segment the image by clustering pixels, performing edge detection, and/or region based (or threshold) segmentation. For example, the segmentation circuit 135 may employ k-means clustering to segment an image into k-objects by clustering similar pixels of the image. The segmentation circuit 135 may also segment an image by performing edge detection. One or more filters/masks may be convolved with the image to detect edges in the image. For example, a sobel filter may be convolved with the image to extract vertical and horizontal edges. The segmentation circuit 135 may also segment an image by comparing each pixel in the image to one or more thresholds. Each threshold may be associated with a class such that when the segmentation circuit 135 determines that a pixel satisfies a particular threshold, the image segmentation circuit 135 determines that the pixel belongs to that particular class. The segmentation circuit 135 may also train a machine learning model to segment an image using supervised learning, for instance. The segmentation circuit 135 may be configured to detect objects (e.g., teeth, gums, lips, tongue, other parts of a mouth) by identifying the coordinates of the objects, rather than painting the detected objects onto a template, as disclosed herein.

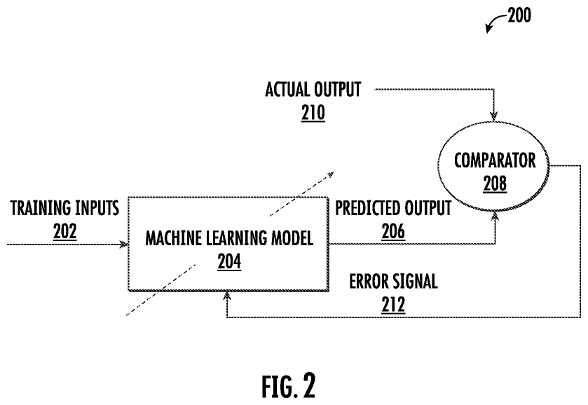

Referring to FIG. 2, a block diagram of an example system 200 using supervised learning that may be used to segment an image is shown according to an example embodiment. Supervised learning is a method of training a machine learning model given input-output pairs. An input-output pair is an input with an associated known output (e.g., an expected output, a labeled output). The machine learning model 204 may be trained on known input-output pairs (e.g., images of teeth and segmented images of teeth) such that the machine learning model 204 learns how to predict known outputs given known inputs. Once the machine learning model 204 has learned how to predict known input-output pairs, the machine learning model 204 can operate on unknown inputs to predict an output.

To train the machine learning model 204 using supervised learning, training inputs 202 and actual outputs 210 may be provided to the machine learning model x200. In some embodiments, training inputs 202 may include an image of a one or more teeth. The training images may be historic images of teeth captured from patients, professional medical imaging equipment, and the like. Actual outputs 210 may include segmented images contouring one or more teeth (or portions of teeth) in the image. The actual outputs 210 may be determined by manual segmentation. For example, one or more highly skilled and trained clinicians, physicians, orthodontists, dentists, technicians and the like may manually delineate (or label) teeth, crowns, roots, arches, regions of teeth, etc., by drawing contours or segmentations on image data (e.g., the training input 202 images). The training inputs 202 and actual outputs 210 may be stored in memory or other data structure accessible by the machine learning model 204. In some embodiments, the machine learning model 204 may be trained using average training data. That is, the image data (e.g., dentition data) associated with multiple users. Additionally or alternatively, the machine learning model 204 may be trained using particular training data. For example, the machine learning model may be trained according to a single user, regional/geographic users, particular user genders, user's grouped with similar disabilities, users of certain ages, and the like. Accordingly, the machine learning model 204 may be user-specific.

In some embodiments, the machine learning model 204 may be trained to segment multiple characteristics in an image (e.g., one or more regions of teeth and arches). In some embodiments, one machine learning model 204 may be trained to segment one characteristic in an image (e.g., one machine learning model 204 segments each tooth, one machine learning model 204 segments regions of a tooth, one machine learning model 204 segments arches, etc.). The machine learning models are trained to segment the image based on the output data used to train the machine learning model. That is, a machine learning model trained to segment each tooth in a plurality of teeth will use manually segmented images of each tooth in a plurality of teeth as actual outputs 210.

The machine learning model 204 may use the training inputs 202 (e.g., images) to predict outputs 206 (e.g., a predicted segmented image), by applying the current state of the machine learning model 204 to the training inputs 202. The comparator 208 may compare the predicted outputs 206 to the actual outputs 210 (e.g., the manually segmented image) to determine an amount of error or differences.

During training, the error (represented by error signal 212) determined by the comparator 208 may be used to adjust the weights in the machine learning model 204 such that the machine learning model 204 changes (or learns) over time to generate a relatively accurate segmented image, using the input-output pairs. The machine learning model 204 may be trained using the backpropagation algorithm, for instance. The backpropagation algorithm operates by propagating the error signal 212. The error signal 212 may be calculated each iteration (e.g., each pair of training inputs 202 and associated actual outputs 210), batch, and/or epoch and propagated through all of the algorithmic weights in the machine learning model 204 such that the algorithmic weights adapt based on the amount of error. The error is minimized using a loss function. Non-limiting examples of loss functions may include the square error function, the room mean square error function, and/or the cross entropy error function.

The weighting coefficients of the machine learning model may be tuned to reduce the amount of error thereby minimizing the differences between (or otherwise converging) the predicted output 206 and the actual output 210. For instance, because the machine learning model is being trained to automatically segment an image given an image, the automatically segmented image will iteratively converge to the manually segmented image. The teeth tracking application 125 may train the machine learning model 204 until the error determined at the comparator 208 is within a certain threshold (or a threshold number of batches, epochs, or iterations have been reached). The trained machine learning model and associated weighting coefficients may subsequently be stored in memory or other data repository (e.g., a database) such that the trained machine learning model may be employed on unknown data (e.g., not training inputs 202). Once trained and validated, the machine learning model 204 may be employed during testing (or inference). During testing, the machine learning model may ingest unknown data to automatically segment the image. For example, during testing, the machine learning model 204 may ingest a user-captured image of teeth and automatically segment the image, indicating one or more regions of the tooth, the crown, the roots, etc.

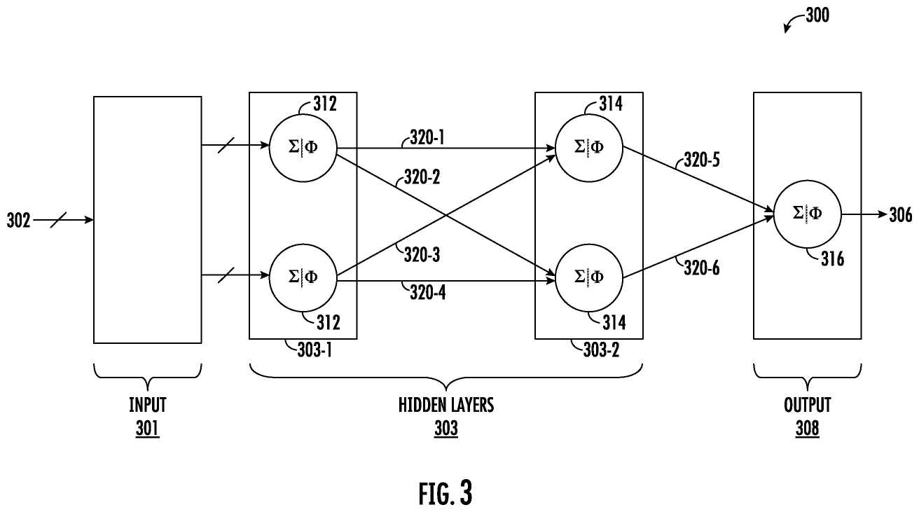

Referring next to FIG. 3, a block diagram of a simplified neural network model 300 is shown, according to an example embodiment. The neural network is an example of the machine learning model (e.g., machine learning model 204 of FIG. 2) that is trained to automatically segment images. The neural network model 300 may include a stack of distinct layers (vertically oriented) that transform a variable number of inputs 302 being ingested by an input layer 301, into an output 306 at the output layer 308.

The neural network model 300 may include a number of hidden layers 303 (or fully connected layers) between the input layer 301 and output layer 308. The hidden layers 303 are fully connected layers because each node 312 in the hidden layer 303-1 is connected to each node 314 in the hidden layer 303-2. In the neural network model 300, the first hidden layer 303-1 has nodes 312, and the second hidden layer 303-2 has nodes 314. The nodes 312 and 314 perform a particular computation and are interconnected to the nodes of adjacent layers. Each of the nodes (312, 314 and 316) sum up the values from adjacent nodes and apply an activation function, allowing the neural network model 300 to detect nonlinear patterns in the inputs 302. Each of the nodes (312, 314 and 316) are interconnected by weights 320-1, 320-2, 320-3, 320-4, 320-5, 320-6 (collectively referred to as weights 320). Weights 320 are tuned during training to adjust the strength of the node. The adjustment of the strength of the node facilitates the neural network's ability to predict an accurate output 306 (e.g., the neural network model 300 ability to learn nonlinear relationships).

In some embodiments, the output 306 may be one or more numbers (e.g., a matrix of real numbers). The one or more numbers or matrix of real numbers may be representative of tooth movements (e.g., a translation/rotation component associated with a final tooth position after treatment).

Referring back to FIG. 1, the segmentation circuit 135 may be configured to receive user inputs from the treatment planning computing system 110. For example, a user, such as a medical professional, may input particular teeth to segment and/or regions of teeth to segment. Accordingly, the segmentation circuit 135 may segment the image according to the user input. In some embodiments, the segmentation circuit 135 may be configured as a segmentation tool, allowing the user to point to regions of interest in the image, delineate regions of interest in the image, and the like.

Referring back to FIG. 1, the teeth tracking application 125 also includes a mapping circuit 115. The mapping circuit 115 may be or include any device(s), component(s), circuit(s), or other combination of hardware components designed or implemented to determine the captured teeth (or regions of teeth) and corresponding views of the captured teeth. Determining a captured tooth may including determining a type of tooth (e.g., incisor, molar, canine, premolars), and a position of the tooth (e.g., upper tooth or lower tooth). Determining the view of the tooth includes determining an orientation of the tooth in the image. For example, the view of the tooth in the image may be from a forward (or front-facing) perspective, a rear (or rear-facing) perspective, a side-facing perspective (e.g., facial perspective, mesial perspective), and the like. In general, the mapping circuit 115 determines (1) whether a tooth (or portion of a tooth) has been captured and (2) what views of the tooth have been captured.

The mapping circuit 115 also paints (or otherwise identifies) the captured teeth and views of the teeth onto a template of a dentition. Additionally or alternatively, the mapping circuit 115 paints (or otherwise identifies) teeth (and/or views of teeth) remaining to be captured on the template based on previously captured teeth. The mapping circuit 115 determines the teeth (and/or view of the teeth) remaining to be captured by determining the inverse of the teeth and/or views of captured teeth on the template. For example, the mapping circuit 115 may compare a blank template to the template painted with the captured teeth (and views of teeth) to determine a template of the remaining teeth (and/or views of teeth) to be captured. The template may include various features of a mouth such as arches, teeth, etc. The template of the dentition may be a generic template, an average template (e.g., a template of teeth across multiple users), a specific template of the user's teeth, and the like.

The mapping circuit 115 may be configured to modify the template based on identifying a flag associated with a user profile and/or a flag associated with a captured image. As described herein, the teeth tracking application 125 may flag the user profile and/or the captured image in response to receiving a message (from the user 120 via the user device 121) related to a tooth characteristic of the user (e.g., the user is missing one or more teeth, missing one or more portions of teeth, has bejeweled teeth, and the like).

In response to receiving a flagged image/user profile, the mapping circuit 115 may query memory 119 for particular user templates to be used as the template model during mapping. For example, the mapping circuit 115 may retrieve a particular user template that indicates the identified one or more missing teeth. In some embodiments, if the flag is encoded with data indicating the location of the missing teeth and/or portions of teeth, the mapping circuit 115 may modify a template (e.g., an average template or a generic template) with the encoded information. That is, the mapping circuit 115 may be configured to remove one or more portions of teeth at locations in the mouth indicated by the encoded information from a template model. Accordingly, the modified template will indicate one or more missing teeth (or portions of teeth). The mapping circuit 115 may associate the user profile with the modified template and store the modified template in memory 119.

As described herein, the mapping circuit 115 may modify the template in response to the user 120 indicating one or more teeth characteristics. Accordingly, the mapping circuit 115 is configured to identify missing teeth in the template. By identifying missing teeth, mapping circuit 115 may differentiate missing teeth from teeth remaining to be captured. When the mapping circuit 115 maps the captured images to the modified template (as described herein), the interactive feedback selection circuit (as described herein) will not select feedback to facilitate the user capturing images of any missing remaining teeth because the missing teeth have been differentiated from the remaining teeth to be captured.

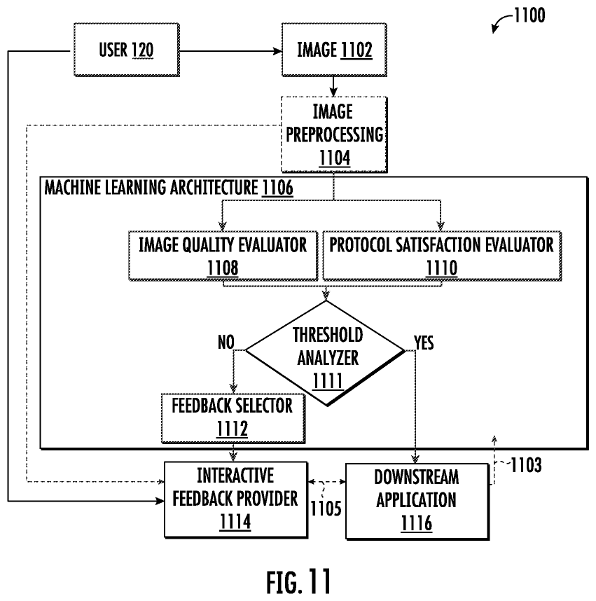

In some embodiments, the mapping circuit 115 may not identify missing teeth. For example, preprocessing operations may not have been performed (e.g., preprocessing operations 1104 in FIG. 11), the segmentation circuit 135 may not have identified any missing teeth, and/or the user profile may not be flagged. In this case, the mapping circuit 115 may compare the cumulatively captured teeth to the template. Accordingly, the mapping circuit 115 may not completely paint the template because the mapping circuit 115 will not map various captured teeth (or portions of captured teeth) to the template. In this case, the teeth tracking application 125 may be configured to receive a user input indicating that the user 120 has captured all of the remaining teeth.

In some embodiments, the mapping circuit 115 may map additional tooth information to the template. For example, metadata determined from the captured images such as a depth of the tooth, the type of tooth, the position of the tooth, the view of the tooth, a focus score of the tooth, and the like, may be mapped to the template.

The mapping circuit 115 determines the captured tooth (and the view of the captured tooth) and identifies the teeth on the template (e.g., maps the captured tooth to the template model) in various ways. In a first embodiment, the mapping circuit 115 paints the captured teeth onto the template by determining a difference between the segmented image of the captured tooth and the template model in a mapped space (e.g., a vector space). In a second embodiment, the mapping circuit 115 paints the captured teeth onto the template by comparing polar coordinates of a tooth object based on the segmented image of the captured tooth to polar coordinates of the template model. In a third embodiment, the mapping circuit 115 paints the captured teeth onto the generic template by mapping the segmented image to the template model using one or more machine learning models.

In the first embodiment, the mapping circuit 115 receives segmented teeth (e.g., from the segmentation circuit 135). The mapping circuit 115 may employ object classification to classify the received segment. The mapping circuit 115 may classify the type of tooth (e.g., incisor, molar, canine, premolars), the position of the tooth (e.g., upper tooth or lower tooth), and/or view of the tooth. In some embodiments, the mapping circuit 115 may employ photogrammetry, as described herein, to determine an orientation of the tooth such that the view of the tooth is represented as an angle.

In an example, the mapping circuit 115 may be a machine learning model trained using supervised learning on inputs of segmented images of teeth and corresponding labeled segmented images of teeth. For example, the labels of segmented images of the tooth can include the type of tooth, the position of the tooth, and the view of the tooth. For example, the labels of teeth can include a front facing top incisor, a mesial facing top incisor, a back of a top incisor, a front facing top canine, etc. The machine learning model of the mapping circuit may 115 may employ a softmax classifier (or any other classifier) to classify the segmented teeth. A softmax classifier uses a softmax function, or a normalized exponential function, to transform an input of real numbers into a normalized probability distribution over predicted output classes. For example, the softmax classifier may predict the probability of the segmented image of the user's teeth belonging to a particular class. The classes are the same as the labels such that the machine learning model learns to classify the images according to the labels. In some embodiments, the mapping circuit 115 may employ SVMs to classify whether the segmented image of the user's teeth belongs to a particular class. Each SVM may be trained to evaluate whether the segmented image belongs to a particular class.

In response to identifying captured regions of teeth (e.g., the tooth type and tooth position) and corresponding views of the captured regions of teeth, the mapping circuit 115 may map the captured teeth into a mapping space. In one embodiment, the mapping space is a vector encoded with information related to the captured teeth and corresponding views of the captured teeth. In one implementation, the mapping circuit 115 may perform one-hot encoding to encode the mapping space vector with the captured teeth and corresponding views. The mapping space vector of encoded data regarding the captured teeth and corresponding angles may be compared to a template vector. The template vector is a vector representing the possible types/positions of teeth in a user's mouth and the corresponding views of each tooth. By comparing the mapping space vector to the template vector (e.g., taking the difference of the vectors), the mapping circuit 115 may determine, with respect to a template model, any remaining teeth to be captured and/or any remaining views of teeth that should be captured. Alternatively, the difference of the mapping space vector and the template vector may indicate all of the regions of teeth that have been captured and the corresponding views of teeth that have been captured.

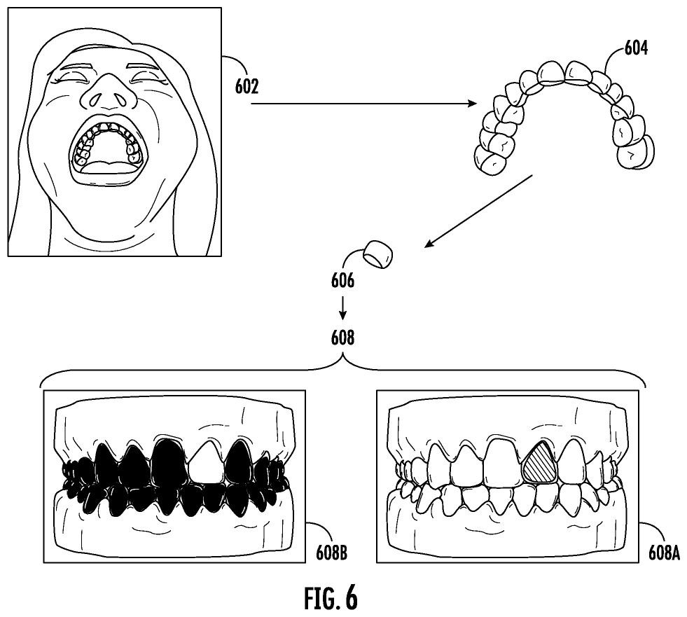

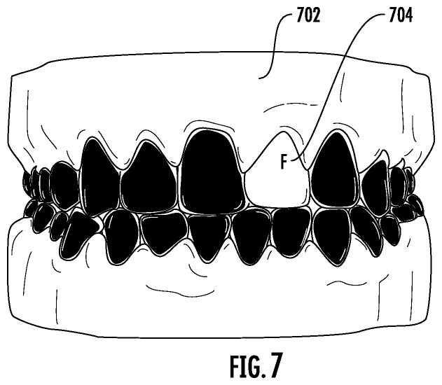

The mapping circuit 115 may subsequently map the results from the difference of the mapping space vector and the template vector to a representation of the template model (e.g., a 3D representation, a point cloud representation, a mesh representation). The mapping circuit 115 paints the template model using the difference of the mapping space vector and the template vector. For example, the mapping circuit 115 may paint a template model to indicate the captured teeth and corresponding views of teeth. For instance, portions of the template model that have been identified as being captured may be painted a color (or the translucency of that tooth of the template model may be adjusted, the brightness may be adjusted, etc.). Additionally or alternatively, the mapping circuit 115 may paint the template model to indicate the remaining teeth to be captured. For instance, the remaining portions of the template model that should be captured may be painted a different color (or the translucency of that tooth of the template model may be adjusted, the brightness may be adjusted, etc.). An example of the mapping circuit 115 painting a template model is described in FIGS. 6-7.

In the second embodiment, the mapping circuit 115 receives segmented teeth (e.g., from the segmentation circuit 135). As described herein, the mapping circuit 115 may employ object classification to classify the received segment. In the first embodiment, the mapping circuit 115 is configured to classify the type of tooth (e.g., incisor, molar, canine, premolars), the position of the tooth (e.g., upper tooth or lower tooth), and/or a view of the tooth (e.g., front-facing perspective, rear-facing perspective, mesial perspective, facial perspective). In the second embodiment, the mapping circuit 115 may be configured to classify only the type of tooth and position of tooth.

The mapping circuit 115 may determine the view of each tooth by transforming the coordinates of the segmented tooth into a different space. For example, a segmented image identified using Cartesian coordinates can be converted to a segmented image identified with polar coordinates using any function or other conversion operation. Each of the segmented teeth (or portions of teeth) received by the mapping circuit 115 may be considered an object in polar coordinate space where each object can be viewed from any direction in the space. The mapping circuit 115 may further ingest orientation parameters and/or the angle of the head of the user including roll, pitch, and jaw angles to determine the view of each tooth visible in an image. The mapping circuit 115 may further determine orientation parameters of the individual jaws including roll, pitch, and jaw angles, where the angles for the upper jaw and the lower jaw may not be the same angle, for example when the mouth is open when the lower jaw is moved relative to the upper jaw.

The mapping circuit 115 may compare the type of tooth and position of the tooth object based on the segmented image to a template tooth object of the same tooth and position. For example, a tooth object of a lower premolar will be compared with a template tooth object of a lower premolar. The mapping circuit 115 determines the view of the tooth object with respect to the template tooth object by comparing the polar coordinates of the tooth object to the template tooth object (e.g., a reference point). Accordingly, the view of the captured tooth is determined based on the angle (in degrees) of the tooth object with respect to the template tooth object. Additionally or alternatively, a range of angles may be mapped to a particular tooth view (e.g., angles between 0° and 25° may be mapped to a front-facing tooth view, angles between 25° and 50° may be mapped to a mesial view, etc.).

In response to identifying the tooth type, tooth position, and view of a segmented tooth (or portion of a tooth), the mapping circuit 115 paints (or otherwise identifies) the corresponding tooth in the template model. In some embodiments, the mapping circuit 115 paints (or otherwise identifies) remaining teeth (e.g., teeth that have not been identified) in the template mode. An example of the mapping circuit 115 painting a template model is described in FIGS. 6-7.

In another embodiment, the mapping circuit 115 may be used to map the captured images to objects or features of the mouth other than a tooth or multiple teeth. For example, the mapping circuit 115 may use a mapping algorithm to determine the coverage of the lips, the tongue, the palate, the gums or other features of the mouth. In one such embodiment, the region of interest may be the palate of the maxilla, and the specific features of interest may be the palatal rugae that should be captured using the application. The mapping circuit 115 can determine the amount and the angle at which images that include coverage of the palate have been captured to establish the overall coverage of the palate.

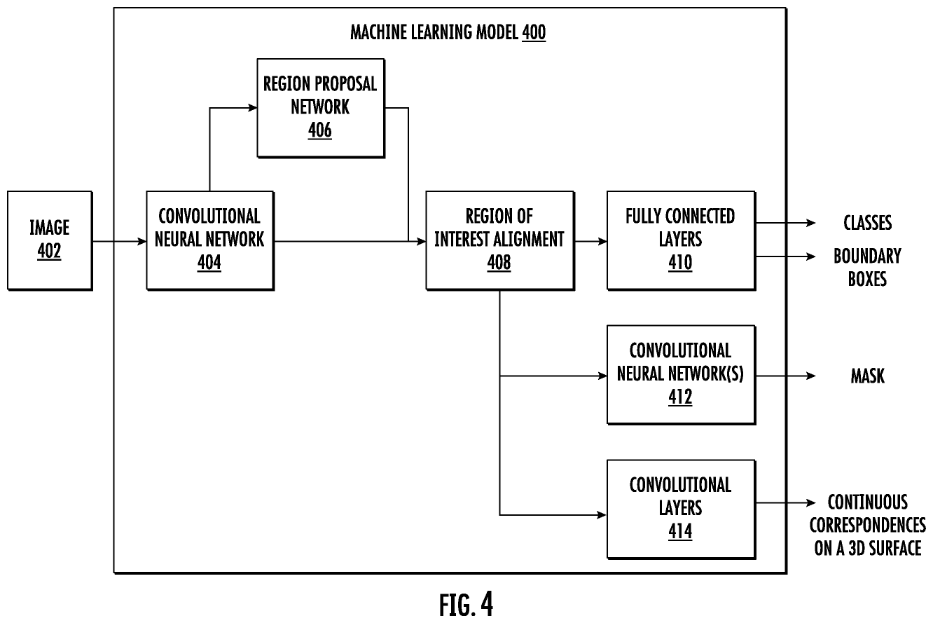

In the third embodiment, the teeth tracking application 125 may feed the captured image to one or more machine learning models as described in FIG. 4. The machine learning model is trained to map the captured 2D image to a 3D representation of the template. Accordingly, the mapping circuit 115 paints (or otherwise identifies) the tooth (or portion of a tooth) identified in the 2D image to a 3D surface. Alternatively, the mapping circuit 115 paints (or otherwise identifies) remaining teeth (e.g., teeth that have not been identified) in the template model based on mapping the teeth that have been captured in the 2D image to the 3D model. In the third embodiment, an AR-generated 3D model, derived from the user's own dental imagery, can be rendered on the user's device, enhancing their understanding of their oral health status. The teeth tracking application 125 can allow the 3D surface to appear in a separate screen segment, track the user's position for interactive experiences, and/or overlay directly on the users' on-screen teeth, creating an immersive and personalized dental visualization. For example, the teeth tracking application 125 can render a three-dimensional surface as an overlay upon the segmented regions of teeth present in the image mapped to the template model.

Referring to FIG. 4, depicted is a machine learning model 400 trained to establish dense correspondences between 2D images (e.g., images of teeth) and 3D models (e.g., the template). The trained machine learning model 400 learns to determine sampled correspondences between segmented 2D images and a 3D surface. For example, the machine learning model 400 may sample points equidistantly in the segmented 2D image. The sampled points of the 2D image are mapped to a 3D representation of the image.

In some embodiments, the machine learning model 400 may be trained using supervised learning in a manner similar to the machine learning model 204 described in FIG. 2. For example, the training inputs of the machine learning model 400 may be annotated 2D images of teeth (or portions of teeth). The actual outputs may be annotated 3D surfaces of a partitioned dentition at various angles. A user, or medical professional, annotates (or labels) correspondences on the 2D image and the partitioned 3D surface. In some embodiments, the medical professional may annotate a predetermined number of rotated views of a particular 3D surface. The predetermined views of the 3D surface beneficially reduces the time it takes the medical professional to annotate the 2D images and 3D surfaces by eliminating a need for the medical professional to manually rotate the 3D surfaces to identify the 2D correspondences. The medical professional responsible for annotating the 2D image and 3D surface may match points on the 2D image to points on any of the predetermined views of the partitioned 3D surface. In some embodiments, when the medical professional annotates a first point on the 3D surface corresponding to the 2D image in one view, the teeth tracking application 125 may render the same point on the 3D surface in the other views (if the point is visible in the other views) based on the surface coordinates of the first point on the 3D surface in the first view.

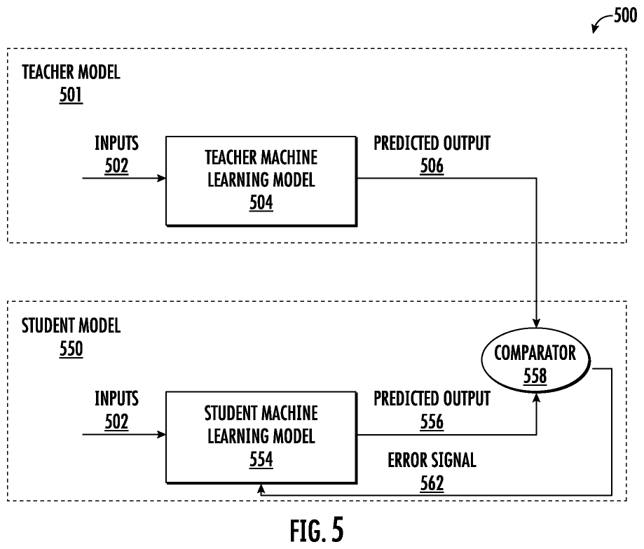

In some embodiments, the machine learning model 400 may be trained using semi-supervised learning. Referring to FIG. 5, a block diagram of an example system 500 using semi-supervised learning that may be used to map pixels of a 2D image to a 3D surface is shown according to an example embodiment. The teacher-student model is an example of a semi-supervised training method. In the teacher-student model, the teacher model 501 is trained on labeled data using supervised learning (as described with reference to FIG. 2). For example, the teacher machine learning model is fed training inputs, where training inputs are annotated 2D images of a dentition (or region of a dentition). In some embodiments, the training inputs may be annotated segmented 2D images. The predicted outputs of the teacher machine learning model are corresponding points on a 3D surface. As described herein with reference to FIG. 2, the comparator (not shown) compares the predicted output to the actual output to determine how accurate the teacher machine learning model is at predicting correspondences between the 2D image and the 3D surface. The error between the predicted output and actual output (determined by the comparator) is fed back to the teacher machine learning model as an error signal to modify the weights in the teacher machine learning model. The modified weights in the teacher machine learning model improve the accuracy of the teacher machine learning model predicting the next input-output pair (e.g., correspondences between the 2D image and the 3D surface). As described herein, the teeth tracking application 125 may train the teacher machine learning model until the error determined at the comparator is within a certain threshold (or a threshold number of batches, epochs, or iterations have been reached). The teeth tracking application 125 stores the trained teacher machine learning model 504 to use to train a student machine learning model 554.

The student model 550 is trained on unlabeled data using psuedolabels generated from the teacher model 501. As shown, inputs 502 are fed to both the untrained student machine learning model 554 and the trained teacher machine learning model 504. The inputs 502 may include labeled input-output pairs (e.g., the training data used for the teacher machine learning model 504 including annotated 2D images of a dentition and corresponding points on a 3D surface) and unlabeled data (e.g., 2D images of a dentition). The student machine learning model 554 determines predicted output 556 based on the inputs 502 by applying the current state of the student machine learning model 554 to the inputs 502. Similarly, the trained teacher machine learning model 504 determines predicted output 506 based on the inputs 502 by applying the trained teacher machine learning model 504 to the inputs 502.

The teeth tracking application 125 trains the student machine learning model 554 using the predicted outputs 506 (or pseudolabels) of the trained teacher machine learning model 504. In cases where the inputs 502 are labeled input-output pairs, the accuracy of the predicted output 506 determined by the teacher machine learning model 504 may be high because the teacher machine learning model 504 was trained using the input-output pairs (e.g., training inputs and corresponding actual outputs, or annotated 2D images of a dentition and corresponding points on a 3D surface).

The comparator 558 compares the predicted output 556 of the student machine learning model 554 to the predicted output 506 of the teacher machine learning model 504 to determine an amount of error or differences. As described herein with reference to FIG. 2, the error (represented by error signal 562) determined by the comparator 558 adjusts the weights of the student machine learning model 554 such that the student machine learning model 554 learns to predict correspondences of both the labeled data (e.g., predicting sampled/annotated correspondences of a 2D image and 3D surface) and the unlabeled data (e.g., predicting continuous correspondences of a 2D image and 3D surface). In this manner, the student machine learning model 554 receives the benefit of learning from the teacher machine learning model 504 by learning relationships determined by the teacher machine learning model 504 and subsequently improving a learned relationship between the input-output pair. Accordingly, the teacher model 501 is used to train the student model 550 to learn how to paint the unlabeled data in the 2D image and predict continuous 3D correspondences.

Referring back to FIG. 4, the trained machine learning model 400 (e.g., student machine learning model 554 in FIG. 5) may be a DensePose-RCNN. As described, the Densepose-RCNN maps a 2D image onto a 3D surface by learning to associate pixels in the 2D image to 3D coordinates. In some embodiments, a segmented image 402 is fed to the machine learning model 400. In some embodiments, the machine learning model 400 may perform image segmentation to extract a tooth (or a region of a tooth) from the image 402.

The machine learning model 400 may extract features from the image 402 using various feature extraction techniques. In some embodiments, the machine learning model 400 employs a feature pyramid network to extract features. In some embodiments, the machine learning model 400 employs a convolutional neural network 404 trained to extract features from the image 402. In some embodiments, the machine learning model 400 employs one or more convolutional layers to extract features from the image 402. Each convolutional layer of the machine learning model 400 extracts and/or evaluates the features of the ingested image 402 by applying various convolution operations. The convolutional layers convolve a filter and/or kernel with the image 402 according to the dimensions and operations of the filter, thereby generating a feature map extracting features from the image 402. Increasing the number of convolutional layers may increase the complexity of the features that may be tracked.

The feature map determined from the convolutional neural network 404 may be ingested by region proposal network 406. The region proposal network 406 may be a neural network trained to ingest feature maps and determine regions associated with likely positions of objects. For example, the region proposal network 406 may determine the likelihood that a proposed region contains the target object and the coordinates of the proposed region. The machine learning model 400 may apply regression to regress the proposed regions. In some embodiments, a selective search may generate regions of interest of the image 402 (as opposed to the feature map determined by the convolutional neural network 404) by combining regions based on color similarity, texture similarity, size similarity, share similarity, and the like.

The machine learning model 400 may perform region of interest alignment 408 using the feature map determined from the convolutional neural network 404 and regions determined by the region proposal network 406. Region of interest alignment 408 creates a fixed size feature map for each region of interest. In some embodiments, the machine learning model 400 may employ pooling layers to reduce the dimensionality of feature maps.

The machine learning model 400 feeds the fixed size feature map for each region of interest to fully connected layers 410. The fully connected layers 410 apply any learned non-linear relationships to the feature maps. The output of the fully connected layers may be fed to classifiers such as support vector machines (SVMs) or a softmax function (described herein) to classify the objects in each region. Each SVM makes a binary determination using a decision boundary that accurately separates linearly separable data while maximizing the margin between the linearly separable data. The SVM determines whether the features of each region belong to the class associated with the SVM. The classifications resulting from the classifier may be used to determine bounding boxes for the classified objects in the regions of interest. The machine learning model 400 may also employ regression to tune the shape of the bounding boxes around each of the objects.

The machine learning model 400 also feeds the fixed size feature map for each region of interest to convolutional neural networks 412. The convolutional neural networks 412 are trained to determine a mask of an object in a region of interest that encodes the spatial layout of the object. Accordingly, the machine learning model 400 determines masks for the objects in the regions of interest in parallel with determining the classification and boundary box of the regions of interest.

The machine learning model 400 also feeds the fixed size feature map for each region of interest to one or more convolutional layers 414. The convolutional layers 414 are configured to predict a gradient of the correspondences of the 2D mapping on the 3D surface. The mapping circuit 115 of FIG. 1 is configured to paint (or otherwise identify) the mapped tooth (or portion of a tooth) identified on the 3D surface based on the 2D image. Alternatively, the mapping circuit 115 paints (or otherwise identifies) remaining teeth (e.g., teeth that have not been identified) or remaining segmented regions of teeth in the template model based on the mapped teeth identified on the 3D surface.

As shown, the machine learning model 400 has a cascading architecture because the output of the region of interest alignment operation 408 is configured to cascade to multiple stages of the machine learning model (e.g., fully connected layers 410, convolutional neural network(s) 412, and convolutional layers 414). In some embodiments, the machine learning model 400 may employ a cross-cascading architecture such that output of one or more stages is fed as an input to a subsequent stage. In this manner, the machine learning model 400 provides subsequent stages context and may determine a refined output.

FIG. 6 illustrates an example of a captured image being mapped to a template model, according to an example embodiment. The teeth tracking application 125 may receive a stream of data recorded from the user device 121. The teeth tracking application 125 samples the data stream for a single frame such that the single frame is processed. In some embodiments, each of the frames of the data stream may be processed. The single frame may be considered an image (e.g., image 602). The segmentation circuit 135 segments the image 602 to generate segmented teeth 604. In some embodiments, the segmented teeth 604 may be further segmented into segmented regions of teeth. As shown, the segmented teeth 604 is further segmented such that a singular tooth is segmented 606. The segmented teeth 604 may include complete or partial views of each tooth. For example, some teeth may be covered by the lips or the tongue, but the parts visible in the image may still be considered. In some embodiments, portions of the singular segmented tooth may be further segmented.

The single segmented tooth 606 may be fed to the mapping circuit 115 for classification/identification as described herein. For example, the mapping circuit 115 may identify the segmented single tooth 606 as a front view of an upper incisor. The mapping circuit 115 maps the tooth (upper incisor) and angle (front view) to the template model based on a mapping approach described herein. For example, the mapping circuit 115 may determine a difference of a template model vector and a mapping vector in a single mapped space (as described in the first embodiment), compare coordinates of the tooth to coordinates in the template model (as described in the second embodiment), or the mapping circuit 115 may use one or more machine learning algorithms such as DensePose (as described in the third embodiment).

In response to the mapping, the teeth tracking application 125 displays a record of the captured teeth on a template model 608. As shown, the record of the captured teeth on template model 608 may be displayed as a record of the captured teeth 608A. Alternatively, by comparing a record of the teeth (or portions of teeth) that have been captured to a template model, the teeth tracking application 125 can convey what is missing (e.g., the remaining teeth or remaining segments of teeth to be captured) as shown in 608B. In some embodiments, the record of the captured teeth/remaining teeth/segments to be captured is feedback that implicitly guides the user to capture images of additional teeth.

In some embodiments, the teeth tracking application 125 may display an indication of the number of angles captured for each tooth. If the teeth tracking application 125 requires four angles per tooth, the teeth tracking application 125 may display a counter (e.g., overlaid on each respective tooth) of the number of angles captured for that particular tooth. The teeth tracking application 125 increments the counter for that particular tooth in response to receiving an image of that tooth at a different angle. In some embodiments, the teeth tracking application 125 may display an arrow, indicating the views of the tooth that have been captured and/or the views of the tooth remaining to be captured. In some embodiments, the teeth tracking application 125 may display a letter representing the views of the tooth that have been captured. For example, FIG. 7 illustrates an example display of a template model and an indication of the views of the tooth that have been captured. As shown, the template model 702 indicates the teeth that have been captured. Further, overlaid on the captured tooth is an indication of the view that has been captured of that tooth 704 (e.g., a front-facing view ‘F’ of the upper incisor has been captured).

Referring back to FIG. 1, in some embodiments, the teeth tracking application 125 may be configured to receive one or more user inputs. For example, the user 120 may interact with the teeth tracking application 125 via a user interface to toggle between the teeth and corresponding angles that have been captured (608A in FIG. 6) and the remaining teeth and corresponding angles to be captured (608B in FIG. 6). The user 120 may also interact with a user interface of the teeth tracking application 125 to toggle a list of the names of the captured teeth and the corresponding captured views and/or a list of the names of the remaining teeth to the be captured and the remaining views. The teeth tracking application 125 may also be configured to receive user inputs that manipulate the template model such that the user 120 is displayed different views of the template model. For example, the user 120 may rotate the template model and identify teeth to be captured and remaining views to capture based on the painted model.

In some embodiments, the mapping circuit 115 is configured to paint the template model according to a confidence of the mapping circuit 115 that the tooth and/or view determined by the mapping circuit 115 using classification techniques, machine learning models, and/or polar coordinates is the correct tooth captured in the image processed by the mapping circuit 115. That is, the mapping circuit 115 may determine a confidence score indicating a likelihood that the identified tooth and/or view in the captured image represents a correct corresponding tooth/view of the user's mouth. The mapping circuit 115 determines the confidence based on algorithmically combining one or more image quality score(s) (e.g., the image quality score determined from the image quality circuit 133 or the image quality score determined from the protocol satisfaction circuit 106) and a confidence associated with the tooth type, tooth position, and/or corresponding view determined from the mapping circuit 115. In an example, if the quality of the image is poor, as determined by the image quality circuit 133 and/or protocol satisfaction circuit 106, then the confidence of the tooth identified by the mapping circuit may be low. For instance, the captured image of the tooth may be blurry (resulting in a low quality score from the image quality circuit 133) or the tooth in the captured image may be blocked by the user's tongue (resulting in a low quality score from the protocol satisfaction circuit 106). Accordingly, the mapping circuit 115 may paint the corresponding tooth/view on the template model a certain low confidence color, brightness, luminosity, pattern, etc. If the quality of the image is high, as determined by the image quality circuit and/or protocol satisfaction circuit, then the confidence of the tooth identified by the mapping circuit may be high. Accordingly, the mapping circuit 115 may paint the corresponding tooth/view on the template model a certain high confidence color, brightness, luminosity, pattern, etc. In some embodiments, the mapping circuit 115 may paint the corresponding tooth/view on the template model a confidence in a range of confidences between a high confidence and a low confidence.

The mapping circuit 115 may increase the confidence that the determined tooth and/or view determined is the correct tooth captured in the image processed by the mapping circuit 115. For example, if the teeth tracking application 125 receives more images of the same tooth and the mapping circuit 115 continues to identify the same tooth/view based on different image qualities (e.g., darkness, blur, lip position) and views, then the confidence that the identified tooth/view is the correct tooth/view in the captured image may increase. It should be appreciated that while the mapping circuit 115 may increase the confidence that the identified tooth/view is the correct tooth captured in the image, the quality of the image may still be low (e.g., the image may be blurry, the tooth may be covered). A high confidence of the tooth/view may be acceptable even if the image quality is low for certain downstream applications. In other downstream applications, high confidence of a low quality image of a particular tooth/view is unacceptable. That is, high quality images, in addition to high confidence associated with various teeth/views are required for some downstream applications.

The mapping circuit 115 may infer that some views of the teeth are visible (and/or some portions of the teeth are visible) and therefore should be painted on the template. Moreover, the mapping circuit 115 may determine that capturing some portions of a dentition might may capturing other portions less necessary to capture. For example, the mapping circuit 115 may determine that capturing a left lower molar may imply that the right lower molar is also captured based on the principals of symmetry.

Feedback (e.g., operator/user instructions) is communicated to the user 120 to increase the probability of a subsequent image (or frame) being a high quality image (e.g., satisfying image quality thresholds where the image quality threshold includes image quality thresholds associated with the characteristics of the image and the image quality thresholds associated with the content of the image). Feedback is also communicated to the user 120 such that a user 120 is guided explicitly on any remaining teeth to be captured (or portions of teeth) and/or any remaining particular views of teeth to capture.

Feedback may be determined using the feedback selection circuit 105. The feedback selection circuit 105 may be or include any device(s), component(s), circuit(s), or other combination of hardware components designed or implemented to determine, identify, or otherwise select (or generate) feedback directed to improving the quality of the image with respect to the content of the image, improving the quality of the image with respect to the characteristics of the image, and also guiding the user to capture remaining teeth and/or particular views of teeth for various downstream applications. The feedback selection circuit 105 can be designed using various state machine frameworks. One such architecture may be an acceptor finite state machine, which focuses on accepting or rejecting input based on defined conditions, thus guiding the user to capture optimal images. Alternatively, a classifier finite state machine could be employed, dividing inputs into several classes and providing distinct feedback depending on the class to which the input belongs. A transducer finite state machine could be used to transform input sequences into output sequences, creating a dynamic feedback loop as the user adjusts their position or action based on the feedback received. Another architecture could be a sequencer finite state machine that can generate a sequence of outputs based on a sequence of inputs, maintaining a historical context of the user's interactions, and providing feedback that evolves along with the user's progress. Additionally, determinism finite state machines can be employed where the next state is determined by the current state and input, giving feedback directly corresponding to the user's immediate action.

As discussed herein, each downstream application may have unique image quality requirements and/or particular teeth/corresponding views of teeth requirements. For ease of description, the feedback determined by the feedback selection circuit 105 is described with respect to selecting feedback to increase the probability of the subsequent image being a high quality image. However, it should be appreciated that the feedback selection circuit 105 may also select feedback to guide the user to capture a particular tooth (or portion of a tooth) and/or a particular view of a tooth. Determining feedback to facilitate the user in capturing high quality images is described in U.S. patent application No. 27/401,053 titled “Machine Architecture for Imaging Protocol Detector,” filed Aug. 12, 2021, the contents of which are hereby incorporated by reference in their entirety.

The flow of information through these state machines can be managed with a variety of methods. One such flow may be a moving window over a probability of high quality images. Alternatively, Kalman Filters, which are optimal recursive data processing algorithms, can be used to estimate the state of a dynamic system from noisy measurements. Additionally, alternative techniques such as Particle Filters could be utilized for non-linear and non-Gaussian processes, Sequential Monte Carlo methods could be utilized for handling complex, high-dimensional systems, Hidden Markov Models could be utilized to account for underlying processes that are not directly observable but can be inferred through outputs. The choice of method can be tailored to the requirements of the feedback system, providing a wide scope for scalability and adaptability in feedback generation.

The feedback may be communicated to the user 120 visually (e.g., on a screen of the user device 121), audibly (e.g., projected from a speaker of the user device 121), using haptics (e.g., vibrating the user device 121), or any combination. In one implementation, the frequency of vibration may decrease (or increase) when the user 120 adjusts the user device 121 closer to a desired location (resulting in a higher quality image). In other implementations, the user feedback (e.g., the feedback communicated to the user) may indicate that the image is not optimal and/or is more optimal/less optimal from the previous image. In some implementations, memory 119 may store various user preferences associated with the user feedback. For example, a user preference may include only providing user feedback displayed on the user device 121 (e.g., not providing audio user feedback). An example of a different user preference may include providing audio user feedback during certain hours of a day (e.g., from 8 AM to 8 PM) and providing haptic feedback during different hours of a day.

The feedback may be provided to the user based on unique user settings. For example, if the teeth tracking application 125 determines that the user 120 has access to hardware (e.g., object detection is used to detect hardware in the image, the user 120 responded to a prompt and indicated that the user 120 had hardware), then the feedback may incorporate the hardware. For example, the feedback may instruct the user to tilt the hardware in a particular direction. In other implementations, the teeth tracking application 125 may be configured to retrieve, from memory 119, one or more videos, instruction manuals, hyperlinks, etc. to be transmitted the user device 121. The videos/instructional manuals/hyperlinks may contain information that explains to the user 120 how to insert the hardware into the user's mouth and/or various tips/tricks associated with using the hardware. The teeth tracking application 125 learns to provide feedback associated with different hardware based on a diverse training set (e.g., receiving images with the hardware, receiving inputs explicitly identifying hardware, and the like). Further, the feedback may be provided to the user 120 based on the region of the user 120, using the language of the user 120, and the like.

In some embodiments, the user may indicate that the user is using a mirror to capture images. For example, the user 120 may employ the rear-facing camera and the mirror to capture images of the user's detention. The user 120 may indicate that they are using the mirror by responding to a prompt asking the user 120 if the user 120 is (or plans to) use a mirror (e.g., that the user 120 intends to face a direction opposite the display of the user device 121). The user may also interact with a button on a user interface displayed on the user device 121, indicating that the user 120 plans/intends to user a mirror to capture images (e.g., that the user 120 intends to face a direction opposite the display of the user device 121).

In some embodiments, the teeth tracking application 125 may also facilitate a “buddy mode” or “assist mode” operation, where another individual assists the user in capturing the images. For example, the teeth tracking application 125 could integrate an option for “buddy mode” at startup, which, when selected, would display a separate set of instructions, tailored to guide an assisting individual on how to position the device for optimal image capture. Furthermore, the application could employ algorithms and sensor data from the user device to provide real-time feedback to the assisting individual. In this mode, a friend, family member, caregiver, dental professional, or dentist, among others, could hold a user device 121 and follow the on-screen instructions to help position the user device 121 and guide the user's actions accordingly. The user's buddy could then provide real-time feedback to the user 120, helping them adjust their mouth position or device placement as necessary for optimal image capture. For example, a “buddy mode” option in the teeth tracking application can be provided, wherein, upon selection of the “buddy mode”, the application provides an alternative set of instructions designed to guide a secondary user in capturing images of the primary user's oral cavity, where the application utilizes face detection algorithms and sensor data from the user device to give real-time feedback to the secondary user, facilitating optimal image capturing and precise representation of the primary user's dental structures. For example, the application could use face detection algorithms to confirm that the user's entire oral cavity is within the camera's view. If not, it could guide the assisting individual with real-time instructions such as “Move closer,” or “Tilt the device downwards,” optimizing the image capturing.

In a different example, the user 120 may turn the user device 121 away from the user 120 such that the display of the user device 121 is not facing the user 120. That is, the display of the user device 121 is not directly visible to the user 120. The teeth tracking application 125 may detect that the user device 121 has been turned using one or more sensors of the user device 121 (e.g., a gyroscope). Responsive to the determination by the teeth tracking application 125 that the user 120 is facing a mirror (or that the display of the user device 121 is not facing the user 120 generally), the teeth tracking application 125 may communicate written/displayed feedback to the user 120 in a mirrored format. The mirrored format is a format of displaying text in a reverse direction such that the text is legible when viewed in a mirror. Accordingly, while the display of the user device 121 is not directly visible to the user 120, a reflection of the display of the user device 121 is visible to the user 120. The teeth tracking application 125 may be configured to generate instructions for the user device 121 to display mirrored text. That is, if the user 120 is viewing the user device 121 through a mirror, the feedback communicated to the user would be legible by the user 120,

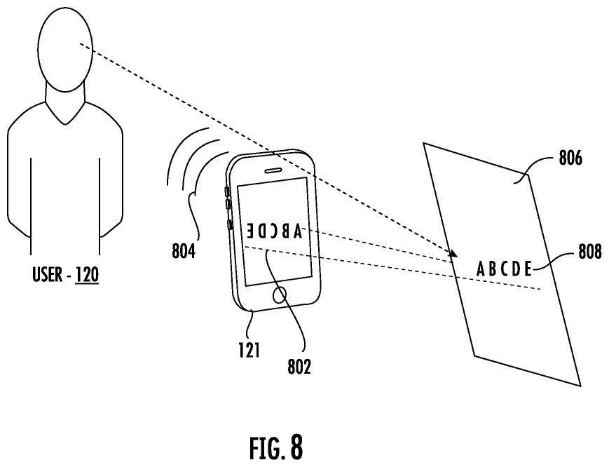

Referring to FIG. 8, a user receiving mirrored user feedback is shown, according to an illustrative embodiment. As shown, the user 120 may use the user device 121 to capture a video of the user's face/mouth (as shown by waves 804). In the illustrative example, the rear-facing camera may be employed to capture the video of the user 120. In response to receiving the video stream, the teeth tracking application 125 on the user device 121 may determine feedback to improve the quality of the image. Determining feedback based on a received image is described herein. As shown, the teeth tracking application 125 determines feedback 802 to improve the quality of subsequently captured video. For illustrative purposes, the feedback to the user 120 is “A B C D E.” The teeth tracking application 125 may also determine, using sensors in the user device 121, that the user 120 is not facing the front of the user device 121 (e.g., the display of the user device 121 is not directly visible to the user 120). Although the display of the user device 121 is not directly visible to the user 120, a reflection of the display of the user device 121 is visible to the user 120 (e.g., via a mirror). Accordingly, the teeth tracking application 125 may generate mirrored feedback 802. The teeth tracking application 125 may use the mirror 806 to display the feedback 802 such that the feedback 802 appears as mirrored feedback 808 on the mirror 806.

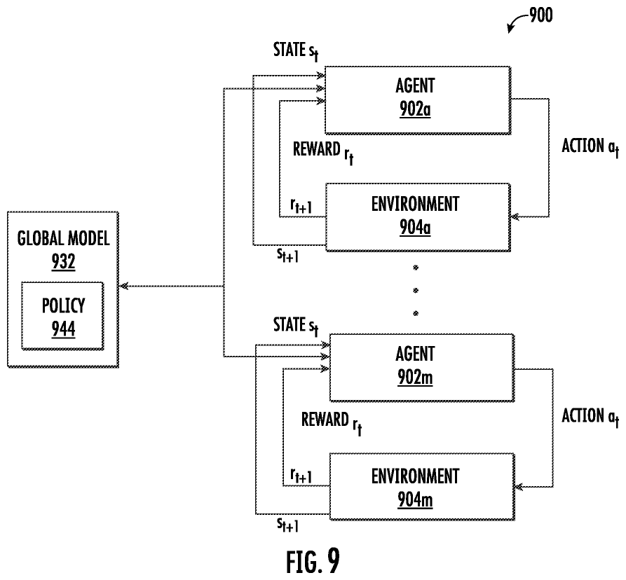

In some embodiments, the feedback selection circuit 105 may include one or more machine learning models. FIG. 9 depicts an agent-based feedback selection model 900 according to an illustrative embodiment. The agent-based feedback selection model 900 may be considered a reinforcement learning model, in which a machine learning model uses agents to select actions to maximize rewards based on a policy network.

Agents 902a to 902m (hereinafter called “agents 902”) refer to a learner or trainer. The environment 904a to 904m (hereinafter called “environment 904”) refers to the quality of the image (e.g., the image characteristics and the image content). At each time step t (e.g., at each iteration), the agent 902 observes a state s t of the environment 904 and selects an action from a set of actions using a policy 944. The policy 944 maps states and observations to actions. The policy 944 gives the probability of taking a certain action when the agent 902 is in a certain state. The possible set of actions include possible user feedback responses. Using reinforcement learning, for example, given the current state of the environment 904, the agent 902 may recommend a particular user feedback or type of user feedback. In some embodiments, if the image quality score is low (e.g., the image quality threshold associated with image characteristics and the image quality threshold associated with the image content are both not satisfied, or the image quality threshold associated with image characteristics and/or the image quality threshold associated with the image content satisfy a low threshold) then agent 902 may learn to recommend a significant user feedback. Significant user feedback is a more extreme version of regular user feedback. An example of significant user feedback may be “open your mouth very wide.” In contrast, regular user feedback (or simply “user feedback”) may be “open your mouth.”

The solution space (e.g., possible set of actions) may be arbitrarily defined and depend on the solution space considerations. For example, the solution space may be discretized such that the possible solutions are fixed rather than on a continuous range. For instance, the action space may include such actions such as: “open your mouth”, “say cheese”, “move your tongue”, “add more light”, and the like. The action space may also include more complex schemes such as dual feedback instructions and/or dual step sizes for an explore/exploit approach. For example, the action space may include multiple feedback instructions such as, “open your mouth wide and add more light”, “please back up and look towards the camera”, and the like. Additionally or alternatively, the action space may include such actions as “please open your mouth a little wider”, “please reduce the intensity of the light a little bit”, “please get much closer to the camera”, and the like.

In some embodiments, the solution space may represent a type of user feedback, and the teeth tracking application 125 may select user feedback randomly or sequentially from a user feedback script (e.g., a dictionary of phrases) associated with the type of user feedback. The user feedback script may be stored in memory 119A of the user device 121 or may be retrieved from memory 119B of the treatment planning computing system 110. The user feedback script may be predetermined phrases and/or instructions to be communicated (audibly, using a display, using haptic feedback, etc.) by the teeth tracking application 125 when the feedback selection circuit 105 selects the particular type of user feedback. The user feedback script may improve the user experience by making the user feedback more relatable and/or user friendly (e.g., heterogeneous) as opposed to homogenous and static. Further, the user feedback script may be specific to the user 120, the user's 120 language, the user's dialect, the user's 120 age group, or other user preferences.

The feedback script associated with the type of user feedback may be categorized (grouped, or clustered) based on the user feedback type. Accordingly, the agent-based feedback selection model 900 selects the type of user feedback based on the environment 904 and policy 944, and the teeth tracking application 125 may select user feedback communicated to the user 120 from the user feedback script.

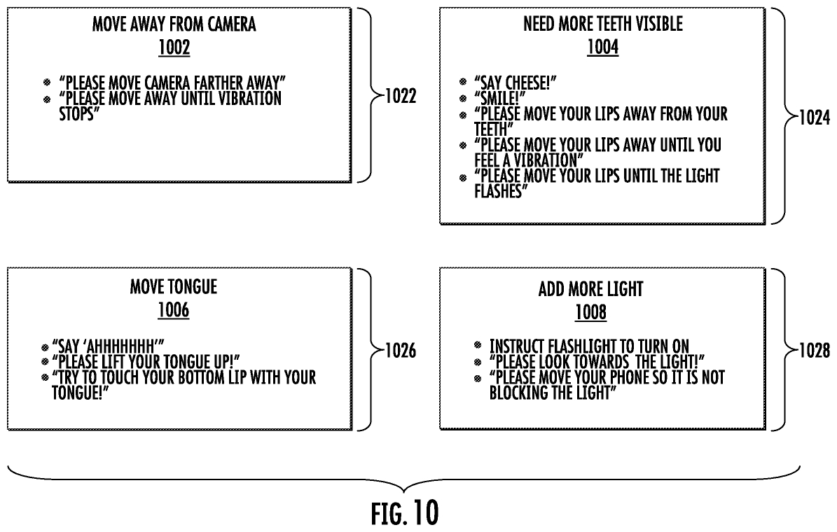

Referring to FIG. 10, illustrated is an example of types of user feedback 1002-1008 and a corresponding user script 1022-1028 for each type of user feedback, according to an illustrative embodiment. For example, a type of feedback selected by the feedback selection circuit 105 may be the “add more light” user feedback type 1008. Accordingly, in response to the user feedback type selected by the feedback selection circuit 105 (e.g., using the agent-based feedback selection model 900), the teeth tracking application 125 selects user feedback communicated to the user 120 from the user feedback script 1028 associated with the user feedback type “add more light”.

For example, using the script 1028 associated with the user feedback type “add more light”, the teeth tracking application 125 may output, using a speaker on the user device 121, “please look towards the light!” Additionally or alternatively, the teeth tracking application 125 may instruct the user device 121 to turn on a flashlight on the user device 121.

Referring back to FIG. 9, the solution space of the agent-based feedback selection model 900 may also be continuous rather than discrete. For example, the action space may include such actions as “move the phone two inches left”, “move the phone 45 degrees right”, “please get 30 centimeters close to the camera”, and the like. In the event a continuous solution space is implemented, the agents 902 may need to train for longer such that the agents 902 can determine, for example, a type of user feedback and a severity (or degree) of change to improve the image quality.

As shown, the agent-based feedback selection model 900 may be an asynchronous advantage actor critic reinforcement learning model. That is, policy 944 is a global policy such that the agents 902 share a common policy. The policy 944 is tuned based on the value of taking each action, where the value of selecting an action is defined as the expected reward received when taking that action from the possible set of actions. In some configurations, the teeth tracking application 125 may update the policy 944 using agents operating in other servers (e.g., via federated learning).

The policy 944 may be stored in a global model 932. Using a global model 932 allows each agent 902 to have a more diversified training dataset and eliminates a need for synchronization of models associated with each agent 902. In other configurations, there may be models associated with each agent, and each agent may calculate a reward using a designated machine learning model.

An agent 902 may select actions based on a combination of policy 944 and an epsilon value representative of exploratory actions and exploitation actions. An exploratory action is an action unrestricted by prior knowledge. The exploratory action improves an agent's 902 knowledge about an action by using the explored action in a sequence resulting in a reward calculation. For example, an exploratory action is selecting a user feedback type that may not have been selected in the past. An exploitation action is a “greedy” action that exploits the agent's 902 current action-value estimates. For example, an exploitation action is selecting a user feedback type that has previously resulted in a high reward (e.g., selecting the user feedback type resulted in a subsequently captured high quality image).

Using epsilon-greedy action selection, for example, the agent 902 balances exploratory actions and exploitation actions. The epsilon value may be the probability of exploration versus exploitation. The agents 902 may perform exploitation actions and exploration actions based on the value of epsilon. The agent 902 may select an epsilon value and perform an exploitation action or an exploratory action based on the value of the epsilon and one or more exploitation and/or exploration thresholds. The agent 902 may randomly select an epsilon value, select an epsilon value from a predetermined distribution of epsilon values, select an epsilon value in response to the number of training epochs, select an epsilon value in response to one or more gradients, and the like. In some embodiments, as training progresses, exploitation actions may be leveraged to refine training. For example, the teeth tracking application 125 may revise the epsilon value (or epsilon selection) such that the likelihood of the exploration action is higher or lower than the likelihood of the exploitation action. Additionally, or alternatively, the teeth tracking application 125 may revise the exploitation action threshold and/or the exploration action threshold.

In response to selecting an action (or multiple actions) according to the epsilon value and policy 944, the environment 904 may change, and there may be a new state sₜ₊₁. The agent 902 may receive feedback, indicating how the action affected the environment 904. In some configurations, the agent 902 determines the feedback. In other configurations, the teeth tracking application 125 may provide feedback. For example, if a subsequent image received by the teeth tracking application 125 is a high quality image, then the teeth tracking application 125 can determine that the action resulting in the subsequent image was an appropriate action. That is, the teeth tracking application 125 may determine a positive reward associated with selecting the action.

The agent 902 learns (e.g., reconfigures its policy 944) by taking actions and analyzing the rewards. A reward function can include, for example, R(sₜ), R(sₜ,aₜ), and R(sₜ,aₜ,sₜ₊₁) In some configurations, the reward function may be a user recommendation goodness function. For example, a reward function based on a user recommendation goodness function may include various quadratic terms representing considerations determined by a trained professional. That is, recommendations and other considerations used by a trained professional may be modeled into a user recommendation goodness function.

Each iteration (or after multiple iterations and/or steps) the agent 902 selects a policy 944 (and an action) based on a current state s t, the epsilon value, and the agent 902 (or the machine learning model 932) calculates a reward. Each iteration, the agent 902 (or machine learning model 932) iteratively increases a summation of rewards. One goal of reinforcement learning is to determine a policy 944 that maximizes (or minimizes) the cumulative set of rewards, determined via the reward function.

The teeth tracking application 125, for instance, weighs policy 944 based on the rewards determined at each step (or series of steps) such that certain policy 944 (and actions) are encouraged and/or discouraged in response to the environment 904 being in a certain state. The policy 944 is optimized by taking the gradient of an objective function (e.g., a reward function) to maximize a cumulative sum of rewards at each step, or after a predetermined number of steps (e.g., a delayed reward).

In some embodiments, the teeth tracking application 125 may inject parameter noise into the agent-based feedback selection model 900. Parameter noise may result in greater exploration and more successful agent-based feedback selection model 900 by adding noise to the parameters of the policy selection.

In some embodiments, the rewards at each step may be compared (e.g., on an iterative basis) to a baseline. The baseline may be an expected performance (e.g., an expected user recommendation type), or an average performance (e.g., an average user recommendation type based on responses of several trained professionals). For example, historic user recommendations may be associated with images received by the teeth tracking application 125. Evaluating a difference between the baseline and the reward is considered evaluating a value of advantage (or advantage value). The value of the advantage indicates how much better the reward is from the baseline (e.g., instead of an indication of which actions were rewarded and which actions were penalized).

In an example of training using agent-based feedback selection model 900, various trained professionals may determine feedback that they would provide to a user associated with various training images. The user feedback determined by the trained professionals may be used as the baseline by the agents 902. The agents 902 may compare the selected user feedback determined using the agents 902 and the policy to the baseline user feedback to evaluate whether the action selected by the agents 902 should be punished or rewarded. In some implementations, the baseline user feedback may be assigned a score (e.g., +1), and other user feedback types may be assigned a score (e.g., using a softmax classifier). The degree of the reward/punishment may be determined based on the difference of the baseline user feedback score and the selected user feedback score.

The teeth tracking application 125 may iteratively train the policy until the policy satisfies an accuracy threshold based on maximizing the reward. For example, the agents 902 train themselves by choosing action(s) based on policies 944 that provide the highest cumulative set of rewards. The agents 902 of the machine learning model (e.g., the agent-based feedback selection model 900 executing in the feedback selection circuit 105) may continue training until a predetermined threshold has been satisfied. For instance, the agents 902 may train the machine learning model until a predetermined number of steps (or series of steps called episodes, or iterations) have been reached. Additionally, or alternatively, the agents 902 may train the machine learning model until the reward function satisfies a threshold value and/or the advantage value is within a predetermined accuracy threshold.

As shown, the teeth tracking application 125 trains the machine learning model (e.g., the agent-based feedback selection model 900 executing in the feedback selection circuit 105) using, for example, asynchronous advantage actor critic reinforcement learning. In some embodiments, the teeth tracking application 125 trains the agent-based feedback selection model 900 using other reinforcement learning techniques.

The teeth tracking application 125 utilizes various asynchronous agents 902a to 902m associated with a corresponding environment to tune a policy 944. The teeth tracking application 125 may employ a GPU, a CPU, or other computing hardware on the device to instantiate multiple learning agents 902 in parallel. Each agent 902 asynchronously performs actions and calculates rewards using a global model (such as a deep neural network). In some embodiments, the policy 944 may be updated every step (or predetermined number of steps) based on the cumulative rewards determined by each agent 902. Each agent 902 may contribute to the policy 944 such that the total knowledge of the model 932 increases and the policy 944 learns how to select user feedback based on an image ingested by the teeth tracking application 125. Each time the model 932 is updated (e.g., after every step and/or predetermined number of steps), the teeth tracking application 125 propagates new weights back to the agents 902 such that each agent shares a common policy 944.

Additionally or alternatively, the feedback selection circuit 105 may employ one or more lookup tables to select a user feedback response (or a type of user feedback). Lookup tables may be stored in memory 119, for example. In some implementations, one or more results of the image quality circuit 133 and/or the protocol satisfaction circuit 106 may map to a user feedback response. For instance, if the image quality circuit 133 determines that the image quality score satisfies a threshold (or satisfies a range), then a user feedback response (or type of user feedback) may be selected using the lookup table.

In an example, a BRISQUE machine learning model employed in the image quality circuit 133 may determine that the image quality in the inside of the user's 120 mouth is 80 (indicating a low quality image). Accordingly, the feedback selection circuit 105 may map the image quality score (and/or the location of the image quality score, such as the inside of the user's 120 mouth) to select user feedback (e.g., using the user feedback script) associated with a type of user feedback (e.g., “add more light”). That is, an image quality score of 80 inside the user's mouth may map to the type of user feedback “add more light.” In a different example, an image quality score of 30 inside the user's mouth (indicating a good high quality image) may map to the type of user feedback “add a little more light.”

In some embodiments, hardware may be used in conjunction with the teeth tracking application 125. For example, object detection circuit may detect objects in video feed and/or detect objects in captured images. The teeth tracking application 125 may determine, based on the detected object, to provide feedback to the user 120 using the detected hardware. For example, a user 120 in possession of a stretching hardware may receive feedback from teeth tracking application 125 on how to better position the stretching hardware (e.g., place lips around the hardware, insert the hardware further into the user's mouth, stick out the user's tongue with the hardware in the mouth).

In some implementations, the teeth tracking application 125 may recommend that the user 120 use hardware to improve the quality of the image (e.g., to improve an image quality score). For example, the teeth tracking application 125 may recommend common household hardware (e.g., spoons, flashlights) to manipulate the environment of the image and/or the user's mouth. Additionally or alternatively, the teeth tracking application 125 may recommend more sophisticated hardware (e.g., a stretcher, such as a dental appliance configured to hold open the user's upper and lower lips simultaneously to permit visualization of the user's teeth and further configured to continue holding open the user's upper and lower lips in a hands-free manner after being positioned at least partially within the user's mouth where the dental appliance includes a handle having two ends and a pair of flanges at each end of the handle).

In some embodiments, the hardware, such as the dental appliance, may be designed with specific attributes or markers to enable the teeth tracking application 125 to compute the user's position with a high degree of accuracy. For example, if the dental appliance is manufactured to a particular specification, the teeth tracking application 125, upon identifying the size of the appliance in the captured image, can calculate the distance between the user and the device. This calculated distance can be utilized to provide feedback to the user regarding their positioning relative to the device. In particular, by comparing the actual size of the appliance to its size in the image, the teeth tracking application 125 can calculate the spatial relationship and provide more precise positioning feedback to the user. Furthermore, the dental appliance could feature identifiable patterns like a QR code or a checkerboard design, which, when detected in the image, can enable the teeth tracking application 125 to compute the angle of the user's head with precision. For example, the identifiable pattern on the dental appliance, when detected and processed by the teeth tracking application 125, can serve as a reference point, allowing the application to calculate the user's head angle based on the observed orientation of the pattern in the captured image.

For example, the dental appliance could be designed with a known dimension or a scannable code such as a QR code. Upon detecting the known dimension of the appliance or the QR code in the image, the teeth tracking application 125 can estimate the distance between the dental appliance and the user 120. This can be achieved by comparing the real-world known dimension of the appliance (e.g., based on the QR code) with its perceived size in the image. This spatial relationship provides an understanding of how far the user 120 is from the camera, aiding in the tracking of the user's teeth or mouth regions. Additionally, the scannable code on the dental appliance can be detected in the image and used as an orientation reference. For example, based on detecting the scannable code, the teeth tracking application 125 can estimate the user's head angle relative to the camera.

In some embodiments, the teeth tracking application 125 can accommodate the use of common household hardware, such as a spoon, coin, or credit card, designed with specific attributes, features, or markers to enable the teeth tracking application 125 to compute the user's position with a degree of accuracy. For example, if the uses a credit card of a standard size, the teeth tracking application 125, upon identifying its size in the captured image, can calculate the distance between the user 120 and the credit card. This calculated distance can be used to provide feedback to the user regarding their positioning relative to the device. Specifically, by comparing the actual size of the credit card to its size in the image, the teeth tracking application 125 can discern the spatial relationship and provide more precise positioning feedback. Additionally, the household hardware could feature identifiable patterns like a checkerboard design, which, when detected in the image, can enable the teeth tracking application 125 to compute the angle of the user's head with precision. For example, the identifiable pattern on the household hardware, when detected and processed by the teeth tracking application 125, can serve as a reference point, allowing the application to calculate the user's head angle based on the observed orientation of the pattern in the captured image.

Additionally or alternatively, the teeth tracking application 125 may prompt the user for information related to available hardware. For example, the teeth tracking application 125 may ask the user 120 whether the user 120 has access to hardware (e.g., spoons, stretchers, flashlights, etc.). The user 120 may respond orally such that a microphone of the user device 121 captures the user's response and/or the user 120 may respond using the screen of the user device 121 (e.g., interacting with a button on a GUI, entering text into a text field).

Particular downstream applications may require specific images of teeth and/or angles at specific image qualities. For example, a downstream application of the treatment planning computing system 110 may generate a 3D model (or other parametric model) of a user's detention using multiple angles of a user's mouth. The downstream application may generate a treatment plan, manufacture aligners, etc. using the model. Accordingly, the teeth tracking application 125 may be configured to capture not only high quality images of the user's mouth, but also a portion of the mouth necessary to generate the model. For example, the teeth tracking application 125 (and specifically the feedback selection circuit 105) may guide the user 120 on how to capture high quality images of the mouth, and also what portions of the mouth to capture.

In a particular example, the teeth tracking application 125 may determine that a front-facing view of a particular tooth has not been captured. As described herein, the teeth tracking application 125 may determine what portions of the teeth have been captured by tracking the portions of the teeth that have been captured and determining the portions of the teeth that have not been captured. Although the teeth tracking application 125 determined that a front-facing angle of a particular tooth has not been captured, the user 120 may instead capture the portion of the tooth at a side angle. As described herein, the teeth tracking application 125 may map the captured portion of the tooth at the side angle to a template such that the teeth tracking application 125 maintains a record of the captured views of teeth. The teeth tracking application 125 may determine that the image of the particular tooth at the side angle is not the image of the particular tooth at the front-facing angle based on analyzing the mapping circuit 115. The teeth tracking application 125 may invoke the feedback selection circuit 105 to select feedback to guide the user 120 to the desired view of the particular tooth. In other implementations, the teeth tracking application 125 may determine that the image of the particular tooth from the side view, while not the image of the particular tooth from the front view, is still a high quality image of the particular tooth at the side view. That is, the image of the user's tooth at the side view may be a high quality image with respect to the image characteristics (e.g., lighting, blur) and with respect to the image content. Accordingly, the teeth tracking application 125 will track the captured image of the particular tooth (e.g., paint the template model to indicate that a high quality image of the side of the user's tooth has been captured). Subsequently, the teeth tracking application 125 may proceed guiding the user 120 to capture a high quality image of the particular tooth from the front view.

FIG. 11 is an interactive communication flow utilizing the teeth tracking application 125 to improve a quality of an image, according to an illustrative embodiment. The teeth tracking application 125 may ingest an image 1102 received from the user device 121. For example, the user 120 may initialize the teeth tracking application and capture a baseline image 1102. Additionally or alternatively, the image 1102 may be a video (e.g., a continuous stream of frames, where each frame may be considered an image).

In some implementations, the teeth tracking application 125 may perform one or more image preprocessing operations 1104 on image 1102. The preprocessing operation 1104 may be configured to standardize and/or normalize the image 1102 such that subsequent processing (e.g., the image quality circuit 133, the protocol satisfaction circuit 106 and/or the feedback selection circuit 105) operates on stable data (e.g., data that is not significantly varied with respect to noise, smoothing artifacts, and the like).

In some implementations, if image preprocessing operation 1104 is employed to preprocess the image, a post-processing engine (not shown) may be employed to modify, correct, adjust, or otherwise process the output data. For example, if an image is converted to greyscale during preprocessing, then the image may be converted back to a color image during post-processing.

Preprocessing operations 1104 may include one or more object detection/classification algorithms to identify various facial features. For instance, the object detection algorithm may be trained to identify teeth, lips, tongue, nose, a chin, ears, and the like. In an example, the user 120 may capture an image 1102 not including a portion of the user's mouth (e.g., the captured image may include the user's ears). Accordingly, the teeth tracking application 125 may execute interactive feedback provider 1114 (employing the feedback selection circuit 105) to select feedback (e.g., using agents 902 in the agent-based feedback selection model 900) indicating that the user 120 should capture a new image and include a portion of the user's 120 mouth.

The facial features may include characteristics of teeth such as missing teeth, chipped portions of teeth, and/or bejeweled teeth. The teeth tracking application 125 may employ preprocessing operations 1104 to identify characteristics of teeth. In an example, a user 120 may capture an image 1102 of the user's detention, where the user 120 is missing a tooth.

If the preprocessing operations 1104 determine that the user 120 is missing the tooth, the teeth tracking application 125 may transmit a prompt to the user device 121 asking the user 120 whether they are missing the one or more teeth. For example, the teeth tracking application 125 may prompt the user for a confirmation that they are missing the particular tooth. In some instances, the teeth tracking application 125 may not receive a response from the user device 121. In this case, the teeth tracking application 125 may retransmit the prompt to the user device 121 asking the user 120 whether they are missing the one or more teeth. In other instances, the teeth tracking application 125 may receive an indication that the user 120 is not missing any teeth. In this case, the teeth tracking application 125 may continue performing other processing operations 1104 and/or may transmit the image 1102 for subsequent processing to the machine learning architecture 1106. Additionally or alternatively, the teeth tracking application 125 may transmit a prompt to the user 120 via the user device 121 to capture the tooth that the teeth tracking application 125 determined was missing but that the user 120 corrected was not missing. That is, the teeth tracking application 125 may request proof that the tooth identified as missing by the preprocessing operations 1104 is in fact not missing.

In some cases, the teeth tracking application 125 may receive a response from the user device 121 confirming (or approving, acknowledging) that the user 120 is missing the tooth identified by the preprocessing operations 1104. If the user 120 confirms that they are missing one or more teeth, the teeth tracking application 125 may flag the captured image 1102 and/or update a data field of the user account associated with the user 120 (e.g., flag the user 120 account). In some embodiments, the flag may encode data indicating the location of the missing teeth and/or portions of teeth based on the object classification performed during the preprocessing operations 1104.

In some embodiments, the teeth tracking application 125 will flag will the captured image 1102 and apply flags to any subsequently captured images until the teeth tracking application 125 is terminated. For example, a downstream application 1116 may receive enough captured images of teeth, prompting the user 120 that they have successfully captured enough teeth and that the user 120 may terminate the teeth tracking application 125.

In some embodiments, the teeth tracking application 125 will flag the captured image 1102 and may flag any subsequently captured images for a predetermined duration of time and/or for a predetermined number of captured images. For example, each of the images captured in two minutes will be flagged. Additionally or alternatively, the next ten captured images will be flagged. In the event the predetermined duration of time expires and/or the predetermined number of captured images has been captured, the teeth tracking application 125 will perform object detection/classification using the preprocessing operations 1104 and evaluate any subsequently captured images for one or more missing teeth.

In yet some embodiments, the teeth tracking application 125 may always flag a captured image. For example, if the user profile is updated to indicate/record that one or more teeth are missing, then anytime the user 120 accesses the teeth tracking application 125, the teeth tracking application 125 may determine, based on the user 120 account, the missing one or more teeth of the user and subsequently not prompt the user 120 to confirm whether they are missing one or more teeth.

The teeth tracking application 125 may also not transmit a prompt to the user 120 via the user device 121 and instead may flag the captured image 1102 and any subsequently captured images for a predetermined duration of time and/or for a predetermined number of captured images.

The preprocessing operations 1104 may also include parsing a video signal into video frames. The frames may be portions or segments of the video signal across the time series. For example, at time t=0, the teeth tracking application 125 may capture a static snapshot of the video data (e.g., a frame), at time t=2, the teeth tracking application 125 may capture another static snapshot of the video data. The time between frames may be pre-established or dynamically determined. The time between frames may be static (e.g., frames are captured every 2 seconds) or variable (e.g., a frame is captured 1 second after the previous frame, a next frame is captured 3 seconds after the previous frame, and the like). In some embodiments, preprocessing operations 1104 include normalizing the image 1102, scaling the image, and/or converting the image into a greyscale image, among others.

In some implementations, preprocessing operations 1104 may include extracting features of the image 1102. The teeth tracking application 125 may perform feature extraction by applying convolution to the image 1102 and generating a feature map of extracted features. Convolving the image 1102 with a filter (e.g., kernel) has the effect of reducing the dimensionality of the image 1102.

Additionally or alternatively, the preprocessing operations 1104 may down sample the feature map (or the image 1102) by performing pooling operations. Pooling is employed to detect maximum features, minimum features, and the like from a pooling window of a predetermined length/duration. In an example, a maximum pooling window is configured to extract the maximum features of the feature map (e.g., the prominent features having higher relative values in the pooling window). In some configurations, the preprocessing operation 1104 may include a flattening operation, in which the teeth tracking application 125 arranges a feature map (represented as an array) into a one-dimensional vector.

Preprocessing operations 1104 may also include performing pose estimation on the image 1102. The teeth tracking application 125 may perform pose estimation using, for instance, bottom-up pose estimation approaches and/or top-down pose-estimation approaches. For example, preprocessing operations 1104 may implement an autoencoder trained to estimate landmarks on an image. The teeth tracking application 125 performs pose estimation to identify localized sets of landmarks in an image or video frame. The sets of landmarks may be considered a landmark model. For example, there may be a landmark model of a face, a landmark model of a mouth, etc.

The landmarks may indicate coordinates, angles, and features relevant to head angles, mouth angles, jaw angles, and/or visibility of the teeth in the image. The pose estimation may be performed at various levels of granularity. For example, in some embodiments, the teeth tracking application 125 may landmark a face. In some embodiments, the teeth tracking application 125 may landmark a chin on the face, a nose on the face, etc.

The teeth tracking application 125 may use landmarks to determine the image quality with respect to the content of the image. For example, the teeth tracking application 125 may determine that an image of a tooth is low quality because the landmark associated with that tooth may not be detected by the teeth tracking application 125. For instance, the teeth tracking application 125 may compare average landmark models of high quality images to landmark models identified in the captured image. The average landmark models may be average landmark models of all users, average landmark models of similar users (e.g., similar users based on a demographic, users of the same age, users of the same gender, users of the same race), or the like. In other implementations, the teeth tracking application 125 may compare a specific user landmark model (e.g., determined using a high quality image captured at a previous point in time such as with certain hardware and/or with assistance from trained professionals) to landmark models identified in the captured image. In this manner, the teeth tracking application 125 may determine whether one or more landmarks of the landmark models are missing. If landmarks are missing, then something may be obstructing where the landmarks should be. For example, if a landmark associated with a tooth is missing from the image, the teeth tracking application 125 may determine that a tongue, lip, or something else may be in front of the tooth, prohibiting the tooth from being captured in the image.

In some embodiments, the teeth tracking application 125 may be configured to receive inputs from the treatment planning computing system 110 regarding one or more preprocessing operations 1104. For example, the teeth tracking application 125 may be configured to receive inputs to smooth, refine, adjust, or otherwise process the image 1102. The inputs may include a selection of a smoothing processing tool presented on a user interface of the treatment planning computing system 110. As a user of the treatment planning computing system 110 selects various portions of the image 1102 using the smoothing processing tool, the teeth tracking application 125 may correspondingly smooth the 3D digital model at (and/or around) the selected portion using preprocessing operations 1104.

Still referring to FIG. 11, in some implementations, the machine learning architecture 1106 may include several machine learning models. For example, as shown, the machine learning architecture 1106 includes the image quality evaluator 1108, the protocol satisfaction evaluator 1110, and the feedback selector 1112. In other implementations, the machine learning architecture 1106 may be a single machine learning model.

In an example implementation, the machine learning architecture 1106 may be a reinforcement learning model such as an agent-based feedback selection model 900. For example, the input to the machine learning architecture 1106 (e.g., the reinforcement learning model) may be the image 1102, and the output of the machine learning architecture 116 may be user feedback and/or types of user feedback (as described herein, with reference to FIG. 9).

Additionally or alternatively, the machine learning architecture 1106 may be a neural network such as the neural network described in FIG. 3. In an example, a neural network trained as the machine learning architecture may receive training inputs such as historic user inputs (e.g., images captured by the teeth tracking application, image captured by a trained professional). For training purposes, the actual outputs received by the neural network may include actual user feedback and/or types of user feedback. The actual user feedback may be determined by one or more trained professionals in response to evaluating the corresponding image. As described herein, the machine learning architecture 1106 may be trained (e.g., as a single model or as multiple models) using average training data. That is, image data (e.g., dentition data) associated with multiple users. Additionally or alternatively, the machine learning architecture 1106 may be trained using particular training data. For example, the machine learning architecture 1106 may be trained according to a single user, regional/geographic users, particular user genders, user's grouped with similar disabilities, users of certain ages, and the like. Accordingly, the machine learning architecture may be user-specific. Accordingly, the neural network may be trained to ingest images 1102 (or preprocessed images in response to preprocessing operations 1104) and output user feedback (e.g., user feedback and/or types of user feedback).

In some embodiments, the machine learning architecture 1106 may call various circuits and employ various evaluators to determine whether a captured image is a high quality image and provide feedback to the user. For example, the machine learning architecture 1106 may call the image quality evaluator 1108 to evaluate the quality of the image 1102 with respect to image characteristics using the results of the image quality circuit 133. The protocol satisfaction evaluator 1110 may evaluate the quality of the image 1102 with respect to the image content using the results of the protocol satisfaction circuit 106.

For example, the protocol satisfaction circuit 106 may determine a size of the user's 120 tooth based on a captured image 1102. The protocol satisfaction evaluator 1110 may determine, based on the size of the tooth in the image 1102 determined from the protocol satisfaction circuit 106, whether the size of the tooth in the image satisfies a tooth size threshold (e.g., an image quality content threshold).

In some implementations, downstream applications 1116 dictate the image quality threshold. In some implementations, there may be multiple image quality thresholds. For example, a first image quality content threshold regarding the size of a tooth may exist if a downstream application involves diagnosing the user 120. Additionally or alternatively, a second image quality content threshold regarding the size of the tooth may exist if a downstream application involves generating a parametric model of the user's tooth. Additionally or alternatively, the protocol satisfaction evaluator 1110 may select an image quality threshold to apply to the results of the image quality circuit based on the downstream application 1116. Accordingly, the image quality evaluator 1108 determines whether the image quality determined by the image quality circuit 133 satisfies one or more thresholds (e.g., one or more characteristics of the image threshold).

The threshold analyzer 1111 may evaluate the outputs of both the protocol satisfaction evaluator 1110 and the image quality evaluator 1108. In some configurations, if both the protocol satisfaction evaluator 1110 and the image quality evaluator 1108 determine that the image is a high quality image (e.g., with respect to the image content and the characteristics of the image respectively), then the downstream application 1116 will receive the image 1102 (or the preprocessed image resulting from the preprocessing operations 1104).

In other configurations, the image 1102 (or the processed image resulting from the preprocessing operations 1104) may be fed to the downstream application 1116, regardless of whether the threshold analyzer 1111 determines that one or more thresholds of either the image quality evaluator 1108 and/or protocol satisfaction evaluator 1110 are satisfied. The downstream application 1116 may also receive data resulting from the machine learning architecture (e.g., image characteristics determined from the image quality circuit 133, results from the image quality evaluator 1108, image content determined from the protocol satisfaction circuit 106, image content from the protocol satisfaction evaluator 1110, and the like). That is, one or more results from the machine learning models of the machine learning architecture 1106 and/or results from the machine learning architecture 1106 may be provided to the downstream application 1116. In some embodiments, the downstream application 1116 receives the one or more results from the machine learning models of the machine learning architecture 1106 and/or results from the machine learning architecture 1106 periodically. In some embodiments, the downstream application 1116 requests such data. In some embodiments, the machine learning architecture 1106 may continuously transmit such data to the downstream application 1116 until the downstream application 1116 transmits a trigger (or other notification/command, indicated by communication 1103) to stop transmitting the one or more results from the machine learning models of the machine learning architecture 1106 and/or results from the machine learning architecture 1106.

The downstream application 1116 may also receive feedback from the interactive feedback provider 1114 (based on the results of the feedback selection circuit 105) indicated by communication 1105. The downstream applications 1116 may also provide information associated with the image quality (including information associated with the image characteristics and/or information associated with the image content) to the interactive feedback provider 1114 indicated by communication 1105. Accordingly, the interactive feedback provider 1114 (and specifically the feedback selection circuit 105) may determine feedback in response to the data communicated by the downstream application 1116. For example, the downstream application 1116 may complete one or more objectives of the downstream application 1116 (e.g., generate a 3D model (or other parametric model) of the user's teeth from a high quality 2D image of the user's teeth). In response to the downstream application 1116 completing the one or more objectives, the interactive feedback provider 1114 may communicate to the user 120 feedback (determined using the data of the downstream application) such as “Capture Successful!”, “Great Job!”, “Stop Capturing”, or “Finished!” (or other phrases of the dictionary of phrases from the user feedback script).

In an illustrative example, the teeth tracking application 125A of the user device 121 may transmit the image 1102 (or portion of the image identified as a high quality portion of the image) to the teeth tracking application 125B of the treatment planning computer system 110. In some embodiments, before the teeth tracking application 125A of the user device 121 transmits the image 1102 to the teeth tracking application 125B of the treatment planning computer system 110, the teeth tracking application 125A may determine whether the image 1102 satisfies one or more additional criteria (e.g., in addition to determining that the image 1102 is a high quality image). For example, the teeth tracking application 125 may perform pose estimation on the image 1102 and determine whether the landmarks identified using pose estimation are suitable for the teeth tracking application 125B of the treatment planning computer system 110 or other downstream applications at the treatment planning computer system 110.

In some embodiments, the teeth tracking application 125A can utilize the outputs of neural network models (e.g., neural network model 300), such as a model identifying the mouth region, to optimize image cropping prior to transmission. For example, the model can focus primarily on regions of high interest, namely the oral cavity in this context, ensuring that the relevant data is preserved while discarding extraneous details. By limiting the image to include only these areas, the file size can be decreased, thereby improving the efficiency of the image transmission to the teeth tracking application 125B on the treatment planning computer system 110. Furthermore, this also minimizes the risk of errors or data corruption during file transfer.

In some embodiments, the machine learning architecture 1106 (or the image quality evaluator 1108 and/or the protocol satisfaction evaluator 1110) may be used to predict an image quality (including image characteristics and/or image content) of a future image (or multiple future images/portions of images). The future image may be an image that has not been captured by the teeth tracking application 125 yet. In these embodiments, the teeth tracking application 125 may anticipate a movement of the user 120 using predicted result(s) of the machine learning architecture 1106 (or the image quality evaluator 1108 and/or the protocol satisfaction evaluator 1110). The anticipated movement of the user 120 may be fed to the downstream application 1116.

In an illustrative example, if a user 120 moves the user device 121 towards a light, a next image (e.g., a future image) may be brighter than the previous image. In some embodiments, the teeth tracking application 125 may predict that the next image will be closer to the light in response to selecting and transmitting user feedback instructing the user to take a brighter picture. The teeth tracking application 125 may detect the trend toward brighter lighting and may anticipate that future image(s), which have not been captured yet, will be brighter than the currently captured image (or other historic images).

Downstream applications 1116 may include applications executed on the user device 121, the treatment planning computing system 110, and/or a third party device. In some embodiments, downstream applications executed on the treatment planning computing system 110 may be applications that may be performed offline or may be associated with high latency (e.g., the user 120 may wait several minutes, hours, days, or weeks before receiving results from the downstream application).

The downstream application 1116 may be an application that incorporate control systems (e.g., using a proportional integral derivative (PID)) controllers. A PID controller may be a controller that uses a closed loop feedback mechanism to control variables relating to the image capture process. For example, the PID controller may be used to control an input/output circuit 128 (e.g., a generate instructions to move or autofocus a camera at the user device 121).

The downstream application 1116 may also be an application that is configured to generate parametric models of one or more teeth (or portions of teeth), including 3D models/reconstructions of the teeth (or portions of the teeth). Generating 3D models from 2D images is described in more detail in U.S. patent application Ser. No. 16/696,468, now U.S. Pat. No. 10,916,053, titled “SYSTEMS AND METHODS FOR CONSTRUCTING A THREE-DIMENSIONAL MODEL FROM TWO-DIMENSIONAL IMAGES” filed on Nov. 26, 2019, and U.S. patent application Ser. No. 17/247,055 titled “SYSTEMS AND METHOD FOR CONSTRUCTING A THREE-DIMENSIONAL MODEL FROM TWO DIMENSIONAL IMAGES” filed on Nov. 25, 2020, where the contents of these applications are incorporated herein by reference in their entirety.

The downstream application 1116 may also generate a treatment plan (e.g., a series of steps used to correct or otherwise modify the positions of the user's teeth from an initial position to a final position or other intermediary positions) using the high quality images captured of the user's teeth (or regions of teeth). For example, the downstream application 1116 may determine a parametric model from the portions of images determined to be high quality portions of images. The downstream application 1116 may manipulate one or more parametric models of individual teeth. The manipulation may be performed manually (e.g., based on a user input received via the downstream application 1116), automatically (e.g., by snapping/moving the teeth parametric model(s) to a default dental arch), or some combination. The manipulation may include lateral/longitudinal movements, rotation movements, translational movements, etc. The result of the manipulated parametric model(s) may result in a final (or target) position of individual teeth of the user following treatment via dental aligners.

The downstream application 1116 may be configured to generate a treatment plan based on the initial position (e.g., the initial position of the user's teeth indicated in the model corresponding to the portions of teeth from the captured high quality image(s)) and the final position (e.g., the final position of the user's teeth following manipulation of the parametric model(s) and any optional adjustments). The downstream application 1116 may also generate one or more intermediate stages of the treatment plan based on the final position and initial position. For example, an intermediate stage may be a stage halfway between the initial positions and the final positions. The treatment plan may consist of an initial stage, based on the initial position of the teeth, a final stage, based on the final position of the teeth, and the one or more intermediate stages. In some embodiments, the downstream application 1116 may employ automated quality control rules or algorithms to ensure that collisions do not occur at any stage, or any collisions are less than a certain intrusion depth (e.g., less than 0.5 mm). The automated quality control rules or algorithms may also ensure that certain teeth (such as centrals) are located at approximately a midline of the detention. The downstream application 1116 may adjust the final position of the teeth and/or the stages of the treatment plan based on an outcome of the automated quality control rules (e.g., to ensure that collisions satisfy the automated quality control rules, to ensure that teeth are located at approximately their intended position, etc.).

The user may view the 3D teeth representation and/or the treatment plan, in addition to other information relating to the treatment plan (e.g., tooth movements, tooth rotations and translations, clinical indicators, the duration of the treatment plan, the orthodontic appliance that is prescribed to achieve the final tooth position (e.g., aligners), the recommended wear time of the appliance to affect the final tooth position). The information related to the treatment plan may be determined based on historic treatment plans. For example, clinical indicators, the duration of the treatment plan, the orthodontic appliance that is prescribed to achieve the final tooth position, and the recommended wear time of the appliance to affect the final tooth position may be determined from a historic treatment plan with a similar initial position and similar final position. Similarly, the information relating to the treatment plan may take into account biomechanical and biological parameters relating to tooth movement, such as the amount and volume of tissue or bone remodeling, the rate of remodeling or the relating rate of tooth movement.

The downstream application 1116 may also be configured to manufacture an aligner or other piece of hardware (e.g., a retainer). The downstream application 1116 may use a final position of the teeth after treatment and/or a treatment plan to fabricate an aligner. In some embodiments, before the aligner is fabricated, the treatment plan may be approved by a remote dentist/orthodontist. For example, a 3D printing system (or other casting equipment) may cast, etch, or otherwise generate physical models based on the parametric models of one or more stages of the treatment plan. A thermoforming system may thermoform a polymeric material to the physical models, and cut, trim or otherwise remove excess polymeric material from the physical models to fabricate dental aligners (or retainers). The dental aligners or retainers can be fabricated using any of the systems or processes described in U.S. patent application Ser. No. 16/047,694, titled “Dental Impression Kit and Methods Therefor,” filed Jul. 27, 2018, and U.S. patent application Ser. No. 16/188,570, now U.S. Pat. No. 10,315,353, titled “Systems and Methods for Thermoforming Dental Aligners,” filed Nov. 13, 2018, the contents of each of which are hereby incorporated by reference in their entirety. The retainer may function in a manner similar to the dental aligners but to maintain (rather than move) a position of the patient's teeth. In some embodiments, the user 120 using the user device 121 and/or an orthodontist/dentist (or other licensed medical professional) may approve of the fabricated dental aligners by inputting, to the user device or treatment planning computing system 110 respectively, an approval/acknowledgement message. In some embodiments, the teeth tracking application 125 may transmit a notification to the user device 121, prompting the user 120 of the user device 121 to execute the teeth tracking application 125 to capture images of the user's teeth periodically during the treatment plan. Accordingly, the teeth tracking application 125 and/or downstream application 1116 may keep a record of the progress of the user's teeth as the teeth move to the final position of treatment.

The downstream application 1116 may also be configured to guide a user in placing an order based on a generated treatment plan and/or proposed aligners/retainers for facilitating the treatment plan. An order may be a transaction that exchanges money from a user 120 for a product (e.g., an impression kit, dental aligners, retainers, etc.). In some embodiments, the downstream application 1116 may communicate prompts to the user device 121 to guide the user 120 through a payment/order completion system. The prompts may include asking the user 120 for patient information (e.g., name, physical address, email address, phone number, credit card information) and product information (e.g., quantity of product, product name). Responsive to receiving a user 120 input (and in some cases a medical user input from the treatment planning computer system 110), the downstream application 1116 may initiate manufacturing (or printing/fabricating) the product. The user 120 may approve/acknowledge the order by interacting with an “Order Now” or “Pay Now” button (or other graphical indicator) on a user interface of the teeth tracking application, interacting with a slider on the user interface of the teeth tracking application, interacting with an object on the user device, making certain gestures and/or communicating audibly. In response to receiving an approval/acknowledgement from the user 120, the downstream application 1116 initiates the product order. The initiated product order is transmitted to other downstream applications 1116 to initiate the fabrication of one or more products (e.g., dental aligners). The initiated product order may also be transmitted to the treatment planning computing system 110 to be stored/recorded and/or associated with a user profile.

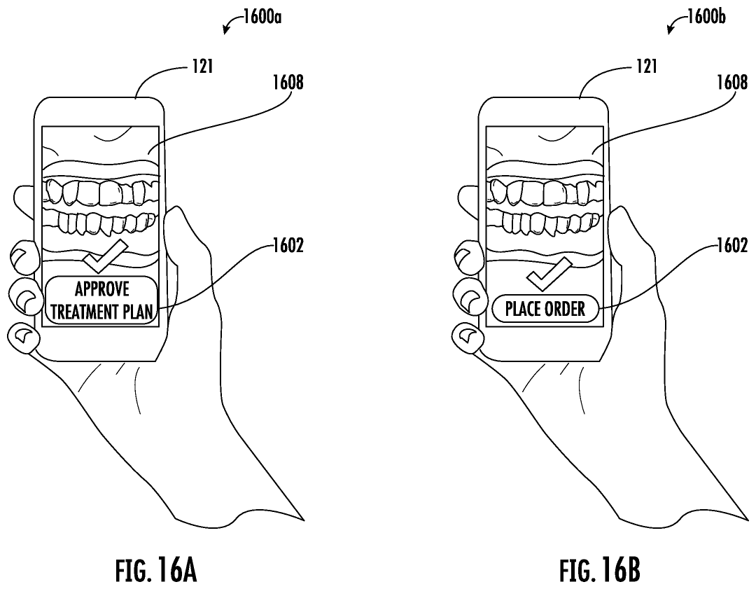

Referring to FIGS. 16A-16B, depicted are examples of a user approving/acknowledging a treatment plan determined by a downstream application. As shown, the user device 121 is displaying a final 3D representation to the user via display 1608. The display 1608 includes interactive button 1602 which indicates that the user approves the final 3D representation of the treatment plan (or intends to place an order). In some implementations, the interactive button 1602 may indicate that the user does not approve of the final 3D representation (e.g., the interactive button may communicate “Don't Like”). In yet other implementations, the interactive button 1602 may be a button (or calendar, automated number dialer, and the like) allowing the user to communicate with an office to book an appointment with a treating dentist, technician, orthodontist, administrator, and the like. In other implementations, the display 1608 may communicate multiple interactive buttons that evaluate whether the user accepts/approves of the final 3D representation of the treatment plan. In some cases, the user may not be able to order/purchase the dental aligners until an administrator has approved the treatment plan. The user's interaction with interactive button 1602 on the user device 121 creates an order and/or purchase that is transmitted to the treatment planning computing system 110 and/or other third party device for storage/subsequent processing of the order/purchase.

The downstream application 1116 may also be configured to monitor a dental condition of the user 120. For example, the downstream application 1116 and/or teeth tracking application 125 may be configured to trigger the teeth tracking application 125 to prompt the user 120 to capture high quality images of the user's teeth at intervals (e.g., annual checks, monthly checks, weekly checks). The downstream application 1116 may scan the high quality image for dental conditions such as cavities and/or gingivitis. For example, the downstream application may use machine learning models trained to classify/identify dental conditions in an image or object detection models to determine whether one or more teeth indicated in a high quality image is affected by a dental condition. The downstream application 1116 may also determine the degree of the dental condition (e.g., a quantitative or qualitative indication of the degree of gingivitis, for instance).

Downstream application 1116 may also monitor a position of one or more teeth of the user 120 by comparing an expected teeth position (e.g., a final position of the treatment plan or other intermediate position of the treatment plan) to a current position of one or more teeth. The downstream application 1116 may monitor the user's teeth to determine whether the user's treatment is progressing as expected. The downstream application 1116 may determine that the user's treatment is progressing as expected by determining that the current position of the user's teeth is within a range of an expected teeth position. For instance, the downstream application 1116 may compare each tooth, or a portion of each tooth, in a current position (including rotational and translational positions) to the corresponding tooth, or portion of each tooth, in an expected position (including rotational and translational positions). The downstream application 1116 may be configured to trigger the teeth tracking application 125 to prompt the user 120 to capture high quality images (or portions of images) of the user's teeth to determine a current position of the user's teeth. In some embodiments, the downstream application 1116 may convert the high quality 2D image of the user's teeth to a parametric model of the user's teeth.

Still referring to FIG. 11, if either the protocol satisfaction evaluator 1110 or the image quality evaluator 1108 determine that the image is not a high quality image, then the interactive feedback provider 1114 may provide feedback to the user 120 (e.g., based on the results of the feedback selection circuit 105). As described herein, the feedback communicated to the user may be audibly communicated to the user, visually communicated to the user (e.g., using a display of the user device 121), communicated to the user using haptic feedback, and the like. In some embodiments, before the feedback is communicated to the user, the teeth tracking application 125 may be configured to check one or more sensors and/or parameters. For example, the teeth tracking application 125 may check the time and communicate feedback to the user based on a time parameter. A time parameter may constrain/limit how the feedback is communicated to the user 120. For instance, between the hours of 9:00 AM and 6:00 PM, the teeth tracking application 125 may communicate feedback audibly. At any other time, the teeth tracking application 125 may communicate feedback visually.

The teeth tracking application 125 may utilize sensors of the user device 121 such as a gyroscope (e.g., to measure angular velocity), an accelerometer (e.g., to measure linear acceleration), and/or one or more cameras to determine orientation information of the user device 121. For example, the teeth tracking application 125 may query a gyroscope of the user device 121 and extract orientation information from the gyroscope information.

The teeth tracking application 125 may also turn on one or more cameras on the user device 121 and determine the orientation of the user 120 with respect to the user device 121 based on which camera (e.g., a front-facing camera or a rear-facing camera) detects/identifies the user. The teeth tracking application 125 may identify/detect the user 120 by analyzing the frames of the front facing camera and determining whether the user 120 is present in the frames associated with the front facing camera. If the user 120 is identified in one or more frames of the front facing camera, the teeth tracking application 125 may determine that the user is in a standard configuration (e.g., the user 120 is capable of viewing the display of the user device 121, or the user is at the front of the user device 121). The teeth tracking application 125 may also identify/detect the user 120 by analyzing the frames of the back facing camera and determining whether the user 120 is present in the frames associated with the back facing camera. If the user 120 is identified in one or more frames of the back facing camera, the teeth tracking application 125 may determine that the user is in a reverse configuration (e.g., the user 120 is not capable of viewing the display of the user device 121).

The teeth tracking application 125 may generate user feedback in response to the determining whether the user is in the standard configuration or the reverse configuration. In response to determining that the user 120 is in the standard configuration (e.g., facing the front of the user device 121, where the front of the user device 121 is the side of the user device 121 with a display), the teeth tracking application 125 may generate instructions to display user feedback using the display of the user device 121. In response to determining that the user 120 is in the reverse configuration, the teeth tracking application 125 may generate instructions to display mirrored user feedback on the display of the user device 121. Accordingly, the feedback displayed on the display of the user device 121 may be understood (e.g., successfully communicated) to the user 120 viewing the display of the user device 121 from the mirror, as illustrated in FIG. 8. In some embodiments, in response to determining that the user 120 is in the reverse configuration, the teeth tracking application 125 may transmit feedback to the user audibly, using haptic feedback, or other feedback mechanisms not relying on the user 120 to view the display of the user device 121 because the display of the user device 121 is not in front of the user 120.

The teeth tracking application 125 may also use one or more cameras (or other sensors) to determine a depth of the teeth. For example, the camera may employ light detection and ranging (LiDAR) to determine the depth of the teeth. The depth of the teeth may be encoded in the captured image as RGBD data. Additionally or alternatively, the camera may indicate a focus score and/or other focus information associated with each captured frame.

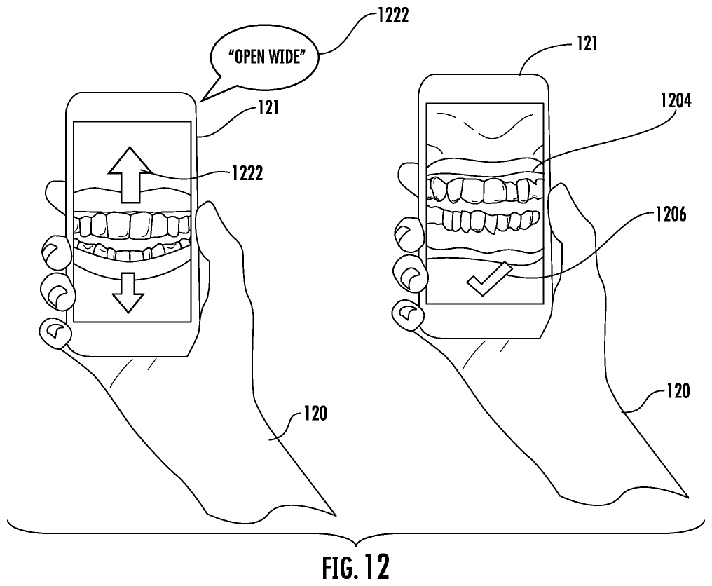

Referring to FIG. 12, illustrated is the interactive communication resulting from the implementation of the machine learning architecture of FIG. 11, according to an illustrative embodiment. The teeth tracking application 125 may receive an image 1102. The teeth tracking application 125 ingests the image and applies the machine learning architecture 1106. The quality of the image is evaluated by the image quality evaluator 1108 (implemented using the image quality circuit 133) to determine whether the characteristics of the image 1102 satisfies one or more thresholds. The image quality evaluator 1108 determines that the image characteristics satisfy the image quality thresholds associated with the image characteristics. The quality of the image is also evaluated by the protocol satisfaction evaluator 1110 (implemented using the protocol satisfaction circuit 106) to determine whether the image content satisfies one or more thresholds. The protocol satisfaction evaluator 1110 determines that the image is not a high quality image based on the image quality score not satisfying an image quality threshold associated with the image content. Accordingly, feedback selector 1112 (implemented using the feedback selection circuit 105) selects feedback to be communicated to the user via interactive feedback provider 1114. The teeth tracking application 125 may determine, before the feedback is communicated to the user 120, that the user device 121 is facing the user 120 in response to determining that the user device 121 is in a standard vertical configuration. As described herein, the teeth tracking application 125 may determine that the user device 121 is in the standard horizontal configuration using orientation information extracted from a gyroscope in the user device 121. As shown, feedback 1222 is both displayed on the user device 121 vertically and audibly announced to the user 120. Feedback 1222 may communicate to the user 120 to adjust the user's lips.

The teeth tracking application 125 receives a subsequent image 1102 from the user 120. The subsequent image is ingested by the teeth tracking application 125 and applied to the machine learning architecture 1106. The quality of the image is evaluated by the image quality evaluator 1108 again (implemented using the image quality circuit 133) to determine whether the image still satisfies the image quality thresholds associated with the image characteristics. The quality of the image is also evaluated by the protocol satisfaction evaluator 1110 again (implemented using the protocol satisfaction circuit 106) to determine whether the image content satisfies the image quality threshold associated with the image content. As shown, responsive to the feedback 1222, the user 120 moves their lips 1204 such that the second image 1102 satisfies the image quality thresholds (e.g., both the image quality thresholds associated with the image characteristics and the image quality thresholds associated with the image content). Indicator 1006 communicates to the user 120 that the second image is more optimal than the first image.

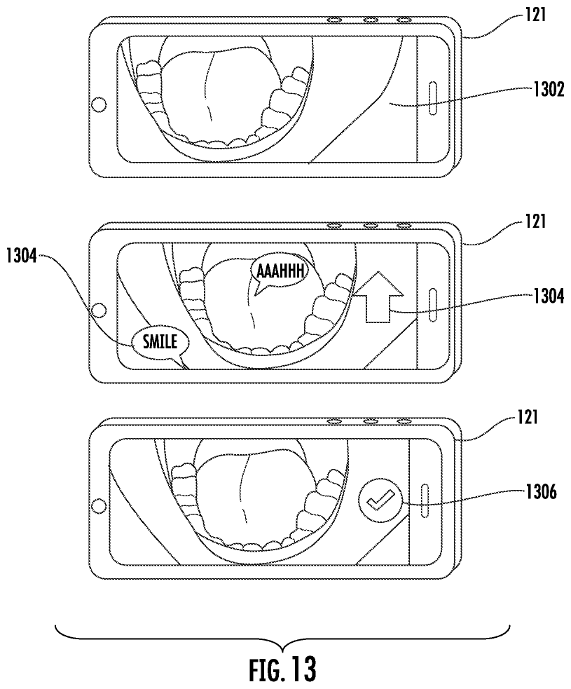

FIG. 13 illustrates the interactive communication resulting from the implementation of the machine learning architecture of FIG. 11, according to another illustrative embodiment. The teeth tracking application 125 may receive an image 1102 as shown in 1302. The teeth tracking application 125 ingests the image and applies the machine learning architecture 1106. The quality of the image is evaluated by the image quality evaluator 1108 (implemented using the image quality circuit 133) to determine whether the image characteristics satisfy one or more thresholds. The image quality evaluator 1108 determines that the image characteristics satisfy the image quality thresholds associated with the image characteristics. The quality of the image is also evaluated by the protocol satisfaction evaluator 1110 (implemented using the protocol satisfaction circuit 106) to determine whether the image content satisfies one or more thresholds. The protocol satisfaction evaluator 1110 determines that the image is not a high quality image based on the image quality score not satisfying an image quality threshold associated with the image content. Accordingly, feedback selector 1112 (implemented using the feedback selection circuit 105) selects feedback to be communicated to the user via interactive feedback provider 1114. The teeth tracking application 125 may determine, before the feedback is communicated to the user 120, that the user device 121 is facing the user 120 in response to determining that the user device 121 is in a standard horizontal configuration. As described herein, the teeth tracking application 125 may determine that the user device 121 is in the standard horizontal configuration using orientation information extracted from a gyroscope in the user device 121. As shown, feedback 1304 is both displayed on the user device 121 horizontally and also audibly announced to the user 120. Feedback 1304 may communicate to the user 120 to adjust the size, distance, angle, and/or orientation of the user device 121 relative to the user 120. Accordingly, the interactive feedback provider 1114 is able to communicate multiple instructions to the user 120 in response to a single input image 1102.

The teeth tracking application 125 receives a continuous data stream (e.g., video data). The teeth tracking application 125 parses the video data into frames and analyzes the frames of the video as if the frames were images. Frames are applied to the machine learning architecture 1106. The quality of the frame is evaluated by the image quality evaluator 1108 (implemented using the image quality circuit 133) to determine whether the image characteristics satisfy the image quality thresholds associated with the image characteristic. The quality of the frame is also evaluated by the protocol satisfaction evaluator 1110 (implemented using the protocol satisfaction circuit 106) to determine whether the image content satisfies the image quality threshold associated with image content. As shown, responsive to the feedback 1304, and based on the continuous adjustments of the user device 121, the teeth tracking application 125 may determine that a frame of the continuous data stream satisfies the image quality thresholds (e.g., both the image quality thresholds associated with the image characteristics and the image quality thresholds associated with the image content). Indicator 1306 communicates to the user 120 that a high quality image has been captured. In some implementations, the teeth tracking application 125 displays the captured high quality image to the user 120.

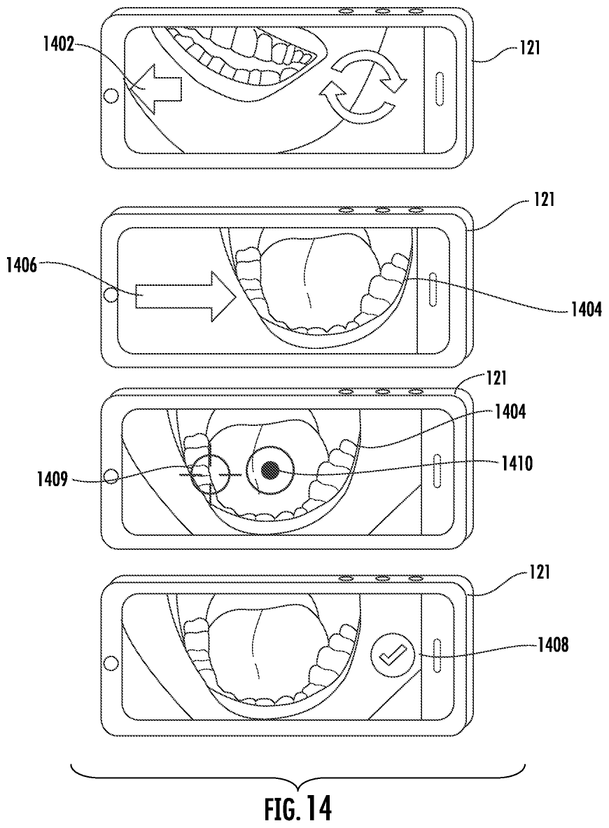

FIG. 14 is an illustration of the interactive communication resulting from the implementation of the machine learning architecture of FIG. 11, according to another illustrative embodiment. The teeth tracking application 125 receives a continuous data stream (e.g., video data). The teeth tracking application 125 parses the video data into frames and analyzes the frames of the video as if the frames were images. Frames are applied to the machine learning architecture 1106. The quality of the frame (image) is evaluated by the image quality evaluator 1108 (implemented using the image quality circuit 133) to determine whether the image characteristics satisfy the image quality thresholds associated with the image characteristics. The image quality evaluator 1108 determines that the image characteristics satisfy the image quality thresholds associated with the image characteristics. The quality of the image is also evaluated using the protocol satisfaction evaluator 1110 (implemented using the protocol satisfaction circuit 106) to determine whether the image content satisfies the image quality threshold associated with the image content. The protocol satisfaction evaluator 1110 determines that the image is not a high quality frame based on the image quality score not satisfying an image quality threshold associated with the image content. Accordingly, feedback selector 1112 (implemented using the feedback selection circuit 105) selects feedback to be communicated to the user via interactive feedback provider 1114. The teeth tracking application 125 may determine, before the feedback is communicated to the user 120, that the user device 121 is facing the user 120 in response to determining that the user device 121 is in a standard horizontal configuration. As described herein, the teeth tracking application 125 may determine that the user device 121 is in the standard horizontal configuration using orientation information extracted from a gyroscope in the user device 121. As shown, feedback 1402 is displayed to the user 120.

In one embodiment, as shown in image 1404, the user 120 responds to the feedback 1402 by opening the user's mouth more, shifting the position of the mouth, adjusting the angle of the mouth, and moving the user device 121 farther away. Continuous streams of data are analyzed by the teeth tracking application 125 resulting in new feedback 1406.

In some embodiments, as shown in image 1404, feedback 1402 can be provided to the user 120 by displaying one or more objects (or symbols, colors) such as a crosshair 1409 and a target object 1410, which are displayed on the user interface of the user device 121. The objects may be any of one or more colors, transparency, luminosity, and the like. For example, crosshair 1409 may be a first color and target object 1410 may be a second, different color. In some embodiments, only one object/symbol may be displayed to the user 120 (e.g., only crosshair 1409 or target object 1410). In some embodiments, both objects/symbols are displayed to the user 120 such that the user 120 is guided to match the objects (e.g., overlay crosshair 1409 onto target object 1410). Continuous streams of data are analyzed by the teeth tracking application 125 resulting in adjusted/moved crosshair 1409 positions and/or target object 1410 positions.

In the event that the teeth tracking application 125 determines that the user device 121 is in a reverse configuration (e.g., the user 120 may be facing the rear facing camera), the feedback displayed may be inverted such that the user 120 viewing the feedback through a reflection of the display of the user device 121 on a mirror will reposition themselves and/or the user device 121 in accordance with the feedback. That is, the user 120 will not decrease the quality of the image by moving themselves and/or the user device 121 in a direction opposite the direction they should move to improve the quality of the image.

The crosshairs 1409 and/or target object 1410 may prompt user 120 to adjust the size, distance, angle, and/or orientation of the user device 121 relative to the user 120 in such a way that the crosshair 1409 is moved toward the target object 1410. The crosshairs 1409 and/or target object 1410 may also prompt user 120 to adjust the user's head, mouth, tongue, teeth, lips, jaw, and the like, in such a way that the crosshair 1409 is moved toward the target object 1410. The target object 1410 can be positioned on the image 1404 relative to an area or object of interest. As the user 120 adjusts the device 121 and/or the user's body, the crosshair 1409 may be moved and positioned such that the adjustment of the user device 121 and/or user 120 by the user 120 increases the image quality score. Additionally or alternatively, the target object 1410 may be moved and positioned such that the adjustment of the user device 121 and/or user 120 by the user 120 increases the image quality score. In one example, the target object 1410 may change into a different symbol or object (e.g., feedback 1408). The target object 1410 may also change colors, intensity, luminosity, and the like. For example, at least one of the crosshair 1409 and target object 1410 may change as the objects become closer to overlapping or once the objects overlap a threshold amount. The crosshair 1409 and the target object 1410 can be overlaid onto the image 1404 using augmented reality methods. The one or more objects (e.g., crosshair 1409 and/or target object 1410) can be placed once or can be repeatedly adjusted during the image capture process.

The teeth tracking application 125 continues to receive continuous data streams (e.g., video data). The teeth tracking application 125 continuously parses the video data into frames and analyzes the frames of the video as images. Frames (images) are applied to the machine learning architecture 1106. The quality of image is evaluated by the image quality evaluator 1108 (implemented using the image quality circuit 133) to determine whether the image characteristic satisfies the image quality thresholds associated with the image characteristics. The quality of the frame is also evaluated by the protocol satisfaction evaluator 1110 (implemented using the protocol satisfaction circuit 106) to determine whether the image content satisfies the image quality threshold associated with the image content. As shown, responsive to the feedback 1406, and based on the continuous adjustments of the user 120/user device 121, the teeth tracking-application 125 determines that a frame (image) of the continuous data stream satisfies the image quality thresholds (e.g., both the image quality thresholds associated with the image characteristics and the image quality thresholds associated with the image content). Feedback 1408 may comprise an indicator that communicates to the user 120 that a high quality image has been captured. In some implementations, the teeth tracking-application 125 displays the captured high quality image to the user 120.

Feedback 1402 and 1406 communicate to the user 120 to adjust the size, distance, angle, and/or orientation of the user device 121 relative to the user 120. Accordingly, the interactive feedback provider 1114 is able to communicate multiple instructions to the user 120.

The interactive feedback provider 1114 may provide a closed feedback loop to the user 120 such that a new image 1102 is captured after the user 120 receives feedback (and responds to the feedback) from the interactive feedback provider 1114. Each of the images 1102 received by the machine learning architecture 1106 are independent. The interactive feedback provider 1114 is configured to provide unique feedback for each image, where each image is captured and analyzed independently of other images. Further, each image may contain a unique set of features.

In response to receiving feedback from the interactive feedback provider 1114, the subsequent image 1102 received by the machine learning architecture 1106 may be improved (e.g., a higher quality image with respect to at least one of the image characteristics of the image or the image content).

Referring back to FIG. 11, in some implementations, regardless of whether the threshold analyzer 1111 determines that image quality thresholds are satisfied, the interactive feedback provider 1112 may be employed to select feedback (using the feedback selection circuit 105) for the user 120 based on the output of the image quality circuit 133 and/or the protocol satisfaction circuit 106. That is, feedback may be provided to the user before the image quality evaluator 1108 and/or the protocol satisfaction evaluator 1110 determine whether image quality thresholds associated with the image characteristics and/or the image content are satisfied.

The image quality evaluator 1108 and protocol satisfaction evaluator 1110 may be machine learning models applied to the same image 1102 in parallel. In some implementations, the user device 121 may apply both the image quality evaluator 1108 and protocol satisfaction evaluator 1110. In other implementations, the user device 121 may apply one machine learning model (e.g., the image quality evaluator 1108) and the treatment planning computer system 110 may apply a second machine learning model (e.g., the protocol satisfaction evaluator 1110).

Additionally or alternatively, the image quality evaluator 1108 and protocol satisfaction evaluator 1110 may be applied to the image in series. For instance, the image quality evaluator 1108 may evaluate the quality of the image using the image quality evaluator 1108 and subsequently evaluate the quality of the image using the protocol satisfaction evaluator 1110 (or vice-versa).

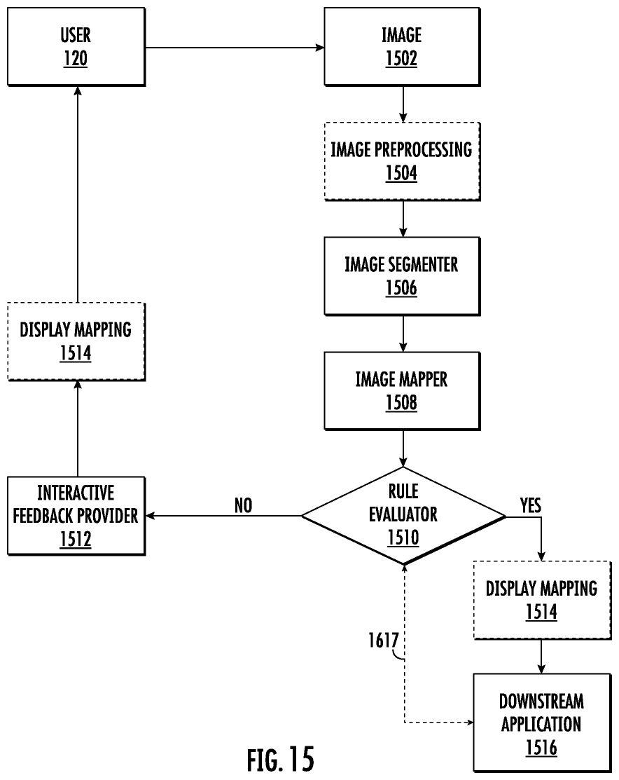

FIG. 15 is an interactive communication flow utilizing the teeth tracking application 125 to track teeth (or portions of teeth) across images, according to an illustrative embodiment. The teeth tracking application 125 may ingest an image 1502 received from the user device 121. For example, the user 120 may initialize the teeth tracking application 125 and capture a baseline image 1502. Additionally or alternatively, the image 1502 may be a video (e.g., a continuous stream of frames, where each frame may be considered an image). In some embodiments, the teeth tracking application 125 may perform image preprocessing operations 1504 (e.g., one or more image processing techniques described with references to FIG. 11). For example, image preprocessing operations 1504 may be configured to standardize and/or normalize the image 1502 such that subsequent processing (e.g., the segmentation circuit 135 and/or the mapping circuit 115) operates on stable data (e.g., data that is not significantly varied with respect to noise, smoothing artifacts, and the like).

The image segmenter 1506 will segment the image 1502. Additionally or alternatively, the image segmenter 1506 will segment the image after preprocessing operations 1504. The image segmenter 1506 may also determine whether the user 120 is missing one or more teeth, in addition to, or instead of, the teeth tracking application 125 determining whether the user 120 is missing one or more teeth based on the preprocessing operations 1504. The image may be segmented by the segmentation circuit 135 to delineate individual teeth, portions (or regions) of individual teeth, an arch, a gum line, fillings, cavities (or other dental conditions), and the like using the segmentation circuit 135.

The image mapper 1508 will map the captured images of teeth to a template model using the mapping circuit 115, creating a record of teeth (or portions of teeth) that have been captured and/or a record of any remaining teeth (or portions of teeth) to be captured.

The rule evaluator 1510 may evaluate the painted template (e.g., map of captured teeth and/or map of remaining teeth to be captured). That is, the rule evaluator may evaluate rules in the mapped space. The rule evaluator 1510 may evaluate predetermined of rules. For example one predetermined rule may involve painting a threshold amount of the template indicating that a threshold number of teeth and/or views of teeth have been captured. For instance, the rule evaluator 1510 may evaluate whether at least 50% of the teeth/views of the template are captured. A different predetermined rule may determine a minimum number of frames for a particular surface. For example, some tooth surfaces may be more important than other tooth surfaces such that a predetermined rule requires a higher minimum number of frames than a number of frames for a different less relevant tooth surface. The rule evaluator 15610 may also evaluate rules received from the downstream application 1516 via communication 1517. As discussed herein, various downstream applications 1516 may perform various operations/functions. Accordingly, each downstream application 1516 may require different teeth to be captured and/or different views of teeth. The rule evaluator 1510 may continue operating the teeth tracking application 125 until the rules (or a portion of the rules) are satisfied.

If the rules are all satisfied (or a portion of the rules are satisfied), then the teeth tracking application 125 may display the mapping 1514. A display of the mapping is described with reference to FIGS. 6-7. The rule evaluator 1510 may also communicate the mapped template, and data associated with the mapped template, to the downstream application 1516. Data associated with the mapped template may include the image 1502, the image after image processing operations 1504 have been applied, the image after the image segmenter 1506 has been applied, each of the captured teeth type (in a vector or other list), each of the captured teeth position (in a vector or other list), each of the views captured, polar coordinates of the captured teeth, 3D representations of the captured teeth, a confidence that each of the mapped teeth in the template are the correct captured teeth, a depth of the captured teeth, and the like.

If one or more predetermined rules are not satisfied, then the interactive feedback provider 1512 may provide feedback to the user 120 (e.g., based on the results of the feedback selection circuit 105). As described herein, the feedback communicated to the user 120 may be audibly communicated to the user, visually communicated to the user (e.g., using a display of the user device 121), communicated to the user using haptic feedback, and the like. In some embodiments, before the feedback is communicated to the user, the teeth tracking application 125 may be configured to check one or more sensors and/or parameters to determine how to communicate the feedback (e.g., displaying text, displaying mirrored text, audibly communicating the feedback, etc.). The feedback communicated to the user 120 is described with reference to FIGS. 12-14. Additionally or alternatively, the template mapped with the captured data (or remaining data) is displayed to the user at 1514. The template model displayed to the user is described with reference to FIGS. 6-7.

In some embodiments, regardless of whether the rule evaluator 1510 determines the predetermined rules and/or rules received from the downstream application 1516 have been satisfied, the interactive feedback provider 1512 may be employed to select feedback (using the feedback selection circuit 105) for the user 120 based on cumulatively captured images mapped to the template.

In some embodiments, based on rules determined by the downstream applications 1516, the rule evaluator 1510 may infer one or more detected portions of the tooth and/or views of the tooth from the captured image and or painted template mode. In a non-limiting example, if a tooth is captured using a bottom view, the incisal edge of the tooth may not be identified in the captured image because the incisal edge is in a wide space. In contrast, if a front view of the tooth is captured, then the incisal edge may be detected and/or inferred from the captured image. In a second non-limiting example, if a side view of a tooth is captured, the interproximal space may not be identified in the captured image. In contrast, if a front view of the tooth is captured, the interproximal space may be identified in the image. Accordingly, the rule evaluator 1510 may determine that more (or less) of the template is captured based on a received image and/or the painted template.

The interactive feedback provider 1512 may provide a closed feedback loop to the user 120 such that a new image 1502 is captured after the user 120 receives feedback (and responds to the feedback) from the interactive feedback provider 1512. In response to receiving feedback from the interactive feedback provider 1512, the subsequent image 1502 received by the teeth tracking application 125 may be a different image of the tooth that furthers (or advances) the mapping of the template. That is, the subsequent image 1502 received may be an image of a different tooth and/or an image of a different view of a tooth.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (e.g., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the Figures. It should be noted that the orientation of various elements may differ according to other exemplary embodiments, and that such variations are intended to be encompassed by the present disclosure.

The hardware and data processing components used to implement the various processes, operations, illustrative logics, logical blocks, modules and circuits described in connection with the embodiments disclosed herein may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit or the processor) the one or more processes described herein.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It is important to note that the construction and arrangement of the systems, apparatuses, and methods shown in the various exemplary embodiments is illustrative only. Additionally, any element disclosed in one embodiment may be incorporated or utilized with any other embodiment disclosed herein. For example, any of the exemplary embodiments described in this application can be incorporated with any of the other exemplary embodiment described in the application. Although only one example of an element from one embodiment that can be incorporated or utilized in another embodiment has been described above, it should be appreciated that other elements of the various embodiments may be incorporated or utilized with any of the other embodiments disclosed herein.

What is claimed is:

1. A system comprising:
a processor and a non-transitory computer readable medium containing instructions that when executed by the processor causes the processor to perform operations comprising:
receiving an image representing a first portion of a mouth of a user;
segmenting the image to generate segmented regions of teeth present in the image;
generating an imaging record by mapping the segmented regions of teeth present in the image to a template model, the imaging record indicating regions of the teeth that remain to be captured; and
providing feedback identifying the regions of the teeth that remain to be captured based on the imaging record.

2. The system of claim 1, wherein mapping the segmented regions of teeth present in the image to the template model comprises determining a difference between the image and the template model in a mapped space, and generating the imaging record comprises determining the regions of the teeth that remain to be captured based on a determined inverse of the segmented regions of teeth present in the image mapped to the template model.

3. The system of claim 1, wherein mapping the segmented regions of teeth present in the image to the template model comprises comparing polar coordinates of a tooth object based on the image and polar coordinates of the template model, and generating the imaging record comprises determining the regions of the teeth that remain to be captured based on a determined inverse of the segmented regions of teeth present in the image mapped to the template model.

4. The system of claim 1, wherein mapping the segmented regions of teeth present in the image to the template model comprises using a machine learning model trained to predict correspondences of a two-dimensional image and a three-dimensional surface, and generating the imaging record comprises determining the regions of the teeth that remain to be captured based on a determined inverse of the segmented regions of teeth present in the image mapped to the template model.

5. The system of claim 1, wherein the feedback comprises remaining views of teeth to be captured, and wherein the feedback is provided to a secondary user based on receiving a selection of an assist mode option on a mobile application, and wherein in response to receiving the selection of the assist mode option, the mobile application provides an alternative set of instructions and feedback to guide the secondary user in capturing images for the imaging record.

6. The system of claim 1, wherein the instructions executed by the processor causes the processor to perform operations comprising:
rendering the three-dimensional surface as an overlay upon the segmented regions of teeth present in the image mapped to the template model; and
communicating user feedback to the user using a display of a user device.

7. The system of claim 1, wherein the feedback is the imaging record, and wherein the instructions executed by the processor causes the processor to perform operations comprising receiving an input toggling the displayed imaging record to display an indication of portions of the mouth captured.

8. The system of claim 1, wherein mapping the segmented regions of teeth present in the image to a template model is based on a confidence score associated with each segmented region of the teeth, the confidence score indicating a likelihood that each of the segmented regions of the teeth have been correctly identified.

9. The system of claim 1, wherein the instructions executed by the processor causes the processor to perform operations comprising:
determining that an image quality score of the image satisfies an image quality threshold, wherein mapping the segmented regions of teeth present in the image to the template model is based on the image quality score satisfying the image quality threshold.

10. The system of claim 9, wherein the image quality score is determined using a machine learning architecture, wherein the instructions executed by the processor causes the processor to perform operations comprising:
providing, based on determining the image quality score, additional feedback to use a piece of hardware to increase the image quality score;
estimating, based on detecting a known dimension of the piece of hardware, a distance between the piece of hardware and the user based on comparing the known dimension of the piece of hardware with a size of at least the portion of the piece of hardware in the image; and
estimating, based on detecting a scannable feature of the piece of hardware, an angle of a head of the user.

11. A computer-implemented method comprising:
receiving, by one or more computer servers having a processor and non-transitory machine readable media, an image representing a first portion of a mouth of a user;
segmenting, by the one or more computer servers, the image to generate segmented regions of teeth present in the image;
generating, by the one or more computer servers, an imaging record by mapping the segmented regions of teeth present in the image to a template model, the imaging record indicating regions of the teeth that remain to be captured; and
providing, by the one or more computer servers, feedback to the user identifying the regions of the teeth that remain to be captured based on the imaging record.

12. The computer-implemented method of claim 11, wherein mapping the segmented regions of teeth present in the image to the template model comprises determining a difference between the image and the template model in a mapped space, and generating the imaging record comprises determining the regions of the teeth that remain to be captured based on a determined inverse of the segmented regions of teeth present in the image mapped to the template model.

13. The computer-implemented method of claim 11, wherein mapping the segmented regions of teeth present in the image to the template model comprises comparing polar coordinates of a tooth object based on the image and polar coordinates of the template model, and generating the imaging record comprises determining the regions of the teeth that remain to be captured based on a determined inverse of the segmented regions of teeth present in the image mapped to the template model.

14. The computer-implemented method of claim 11, wherein mapping the segmented regions of teeth present in the image to the template model comprises using a machine learning model trained to predict correspondences of a two-dimensional image and a three-dimensional surface, and generating the imaging record comprises determining the regions of the teeth that remain to be captured based on a determined inverse of the segmented regions of teeth present in the image mapped to the template model.

15. The computer-implemented method of claim 11, wherein the feedback comprises remaining views of teeth to be captured.

16. The computer-implemented method of claim 11, further comprising communicating user feedback to the user using a display of a user device.

17. The computer-implemented method of claim 11, wherein the feedback is the imaging record, the method further comprising receiving an input toggling the displayed imaging record to display an indication of portions of the mouth captured.

18. The computer-implemented method of claim 11, wherein mapping the segmented regions of teeth present in the image to a template model is based on a confidence score associated with each segmented region of the teeth, the confidence score indicating a likelihood that each of the segmented regions of the teeth have been correctly identified.

19. The computer-implemented method of claim 11, further comprising:
determining that an image quality score of the image satisfies an image quality threshold, wherein mapping the segmented regions of teeth present in the image to the template model is based on the image quality score satisfying the image quality threshold, and wherein the image quality score is determined using a machine learning architecture.

20. A computer-implemented method comprising:
receiving, by a teeth tracking application operating on a user device associated with a user and from a capture device of the user device, an image representing a first portion of a mouth of a user;
segmenting, by the teeth tracking application, the image to generate segmented regions of teeth present in the image;
generating, by the teeth tracking application, an imaging record by mapping the segmented regions of teeth present in the image to a template model, the imaging record indicating regions of the teeth that remain to be captured; and
providing, by the teeth tracking application on a display of the user device, feedback to the user identifying the regions of the teeth that remain to be captured based on the imaging record.
